(19) United States
(12) Patent Application Publication
(10) Pub. No.: US 2007/0275060 A1
(43) Pub. Date: Nov. 29, 2007

(54) Extended release solid pharmaceutical composition containing carbidopa and levodopa

(75) Inventors: Marcelo F. Befumo, Buenos Aires (AR); Marcelo A. Ricci, Buenos Aires (AR); Ethel C. Feleder, Buenos Aires (AR); Glenn A. Meyer, Wilmington, NC (US); Joaquina Faour, Buenos Aires (AR); Juan A. Vergez, Buenos Aires (AR)

Correspondence Address: INNOVAR, LLC, P O BOX 250647, PLANO, TX, 75025, US

(73) Assignee: OSMOTICA COSTA RICA SOCIEDAD ANONIMA, San Jose, CA (US)

(21) Appl. No.: 11/498,307

(22) Filed: Aug. 2, 2006

Related U.S. Application Data
(60) Provisional application No. 60/705,839, filed on Aug. 5, 2005.

(51) Int. Cl. A61K 31/137; A61P 25/16; A61K 9/24

(52) U.S. Cl. 424/468; 424/471; 424/474; 514/649

(57) ABSTRACT

The invention provides a compressed tablet that provides a extended release tablet containing a extended release form of carbidopa and a extended release form of levodopa. The tablet optionally further comprises an immediate or rapid release composition of carbidopa and/or levodopa. The extended release composition in the tablet excludes a release rate-controlling polymer, and a release rate-controlling coating; however, the release of the carbidopa and/or levodopa is independently optionally delayed for a lag time. The invention also provides a tablet having a extended release form of levodopa and a rapid or immediate release form of carbidopa. A tablet can contain levodopa present in extended release form and rapid or immediate release form, and carbidopa present in extended release form and rapid or immediate release form. The tablet is used to treat Parkinson's disease and other movement related disorders, diseases or syndromes.

### CROSS-REFERENCE TO EARLIER FILED APPLICATION

The present application claims the benefit of priority of and is a continuation-in-part of U.S. Provisional Application Ser. No. 60/705,839 filed Aug. 5, 2005, the entire disclosure of which is hereby incorporated by reference.

### FIELD OF THE INVENTION

The present invention concerns a solid oral dosage form that provides an extended release of levodopa and carbidopa. In one embodiment, the invention concerns a pharmaceutical composition providing an extended release of levodopa and carbidopa, over an about 1 to 4 hour period following exposure to an aqueous environment. The invention also provides dosage forms that present a dual release of levodopa and/or carbidopa. In other embodiment the dosage form provides an extended release of levodopa and optionally carbidopa, and an immediate or rapid release of carbidopa. In yet another embodiment the dosage form provides a delayed and extended release of levodopa and optionally carbidopa, and an immediate or rapid release of carbidopa.

### BACKGROUND OF THE INVENTION

Solid dosage forms containing a combination of levodopa (LD) and carbidopa (CD) are well known. Such dosage forms are used to treat Parkinson's disease and other movement disorders. The combination of LD and CD remains the most effective anti-Parkinson drug. Levodopa, the levorotatory isomer of dihydroxy-phenylalanine, is the natural metabolic precursor of dopamine (DA), which is responsible for the therapeutic effectiveness in the central nervous system (CNS). However DA is neither absorbed from the gastrointestinal tract nor able to pass across the brain-blood barrier. Therefore, only LD can reach the CNS. When orally administered, LD is rapidly absorbed by the saturable amino-acid active transport system located in proximal one third of the small intestine (duodenum and jejunum). Peak concentrations are between 30-120 minutes, depending mainly on the gastric emptying time and the length of time the drug is exposed to the degradative enzymes of the gastric and intestinal mucosa. When LD is taken alone, it is metabolized by L-aminoacid decarboxylase (AADC) to DA by the gut mucous, intestinal microflora and liver, causing side effects such as nausea, vomiting, and appetite loss. Carbidopa blocks AADC outside the brain allowing more LD to enter the brain without causing nausea, vomiting, and appetite loss.

SINEMET™ (Bristol Meyers Squibb) is a rapid release oral tablet containing CD (10-25 mg) and LD (100-250 mg). This tablet comprises cellulose, magnesium stearate and starch. ENDO produces a product meeting the same specifications. ATAMET® (ELAN) is a rapid release tablet containing CD (25 mg), LD (100-250 mg), microcrystalline cellulose, magnesium stearate, pregelatinized starch and corn starch. PUREPAC makes a product meeting the same specifications. PARCOPA™ (SCHWARZ) is a buccal release tablet comprising CD, LD, aspartame, citric acid, crospovidone, magnesium stearate, mannitol, microcrystalline cellulose, sodium bicarbonate and mint flavor. Carbidopa/levodopa generic rapid release tablets are also available from SANDOZ and TEVA. None of those tablets are controlled release tablets.

The “wearing-off” and “on-off” phenomena have emerged as major problems in the LD-CD long-term treatment of Parkinson's disease. Within two to five years of initiating combination therapy certain limitations become apparent as the disease progresses, the benefit from each dose becomes shorter (“the wearing off effect”) and some patients alternate unpredictably between mobility and immobility (“the on-off effect”), which may occur many times a day. “On” periods are usually associated with high or rising plasma LD concentrations and often include abnormal involuntary movements, i.e., dyskinesias. “Off” periods have been correlated with low or falling plasma LD levels and bradykinetic episodes. In an effort to reduce the occurrence of “wearing off” and “on-off” phenomena, controlled release oral dosage combinations were introduced. Exemplary ones include SINEMET™ CR (or NACOM™), which is an extended release oral tablet containing CD (25-50 mg) and LD (100-200 mg). It releases drug over a period of about 3-6 hours (Physicians' Desk Reference 57th Ed. 2003 pg. 1111, the disclosure of which is hereby incorporated by reference), which period exhibits inter-patient variability. The tablet comprises hydroxypropylcellulose, polyvinylacetate-crotonic acid copolymer, magnesium stearate, and red ferric oxide. The SINEMET CR tablet is a polymeric-based drug delivery system that controls the release of carbidopa and levodopa as it slowly erodes. The 25-100 (CD-LD) tablet and the 50-200 (CD-LD) tablet each contains the drugs present in 1-4 weight ratio. The interval between doses of SINEMET CR is about 4-8 hours during the waking day; however, it can be administered more frequently and in higher doses if needed. SINEMET CR can be administered in combination with SINEMET immediate release. However, patients taking SINEMET CR can develop increased dyskinesia as compared to SINEMET immediate release. Dempski et al. (Neurology (1989); 39(Suppl. 2):20-4) disclose the results of a study on the pharmaceutical design and development of the SINEMET CR erosion-controlled or diffusion-controlled release formulation. Data from clinical trials cited in the U.S. Pat. No. 4,900,755 indicate that effective antiparkinson effects were achieved with fewer daily doses of the controlled release form as compared with the conventional combination. Related U.S. Pat. No. 4,832,957 discloses a matrix tablet comprising “a polymer vehicle comprising 5-25 mg of water-soluble hydroxypropyl cellulose polymer, and 2-50 mg of a less water-soluble polyvinyl acetate-crotonic acid copolymer.” Related U.S. Pat. No. 4,983,400 discloses a controlled release solid oral dosage formulation for the controlled release of CD and LD consisting essentially of a uniform dispersion of 5-300 mg of CD, 20-1200 mg of LD in a polymer vehicle of 2 to 120 mg of polymethyl methacrylate. This formulation contains poly(methyl methacrylate) as a release rate-controlling polymer.

Generic extended release tablets containing CD (25-50 mg) and LD (100-200 mg) are sold by MYLAN, KV PHARM, IMPAX LABS, and TORPHARM. APOTEX, a Canadian pharmaceutical company, sells a controlled-release formulation of levodopa and carbidopa (APO-LEVOCARB CR tablets) that contains a polymer-based drug delivery system that controls the release of LD and CD as it slowly erodes.

The combination of immediate release and sustained release LD and CD has been under evaluation for long time. Cedarbaum et al. (J Neural Transm Park Dis Dement Sect (1990), 2(3):205-13) disclose the results of long-term treatment with controlled-release levodopa/carbidopa (SINEMET CR) compare to standard SINEMET. The study shows that most patients required standard SINEMET at least one dose each day to hasten the onset of antiparkinson effect and that SINEMET CR is a useful adjunct in the long-term management of motor response fluctuations in Parkinson's disease. LeWitt et al. (Neurology (1992); 42(suppl 1):29-32) disclose clinical studies with and pharmacokinetic considerations of sustained release LD. The authors concluded that the best therapeutic strategy to improved “on” times in parkinsonian patients may be a combination of the conventional SINEMET and SINEMET CR, conventional SINEMET as a booster dose and SINEMET CR for more sustained effect. Stocchi et al. (Clin Neuropharmacol (1994), 17 Suppl 2:S7-13), disclose the clinical implications of sustained dopaminergic stimulation. The study shows that the combination of standard SINEMET and SINEMET CR ensures a more prolonged clinical effect with a very short latency to the “on” phase.

PCT International Publication WO 94/06416 discloses a tablet consisting of a first layer containing one or more drugs with immediate or controlled release formulation comprising polymeric substances which swell or solubilize when contacted with aqueous liquids, wherein the polymeric substances are selected from the group consisting of cross-linked polyvinylpyrrolidone, low and medium molecular weight hydroxypropyl cellulose and hydroxypropyl methylcellulose, cross-linked sodium carboxymethylcellulose, carboxymethyl starch, potassium methacrylate-divinylbenzene copolymer, polyvinyl alcohols, starches, microcrystalline cellulose and β-cyclodextrin; a second layer containing one or more drugs, either equal to or different from those of the first layer, with slow release formulation, comprising polymeric substances which swell or erode or are gellable when contacted with aqueous liquids, wherein the polymeric substances are selected from the group consisting of hydroxypropyl methylcellulose having a molecular weight from 1000 to 4,000,000, hydroxypropyl cellulose having molecular weight from 2,000 to 2,000,000, carboxyvinyl polymers, polyvinyl alcohols, glucans, scleroglucans, mannans, xanthans, alginic acid, carboxymethylcellulose, poly(methyl vinyl ethers/maleic anhydride), ethylcellulose and methylcellulose; and a third layer, which is a low-permeability barrier coating said second layer. The inventors disclose that the availability of pharmaceutical compositions capable of liberating different drugs at successive times would be useful in the case that the drugs benserazide or CD should be administered with or before L-dopa administration; thus the peripheral conversion of L-dopa into dopamine would be drastically reduced and higher amounts of L-dopa would reach the systemic circulation and the brain, where conversion into dopamine produces the desired therapeutic effect. Thus, much lower L-dopa doses can have a high therapeutic effect and, at the same time, produce lesser side effects. There is no LD and/or CD exemplary formulation disclosed. This tablet contains a release rate-controlling polymer.

PCT International Publication WO 99/17745 discloses a controlled-release monolithic system for oral administration. The system comprises a disintegrating layer, an erodible layer and a swelling layer, of which two are external and one is intermediate, each layer containing one or more drugs. In one embodiment the system comprises a swelling layer comprising levodopa methylester, CD, Eudralack red, hydroxypropyl methylcellulose (Methocel K15M), hydroxypropyl methylcellulose phthalate (HPMCP 50), triacetin, talc and magnesium stearate; an erodible layer comprising levodopa methyl hydrochloride, CD, potassium metabisulfite, blue lake, glyceryl palmitostearate, lactose, polyvinylpyrrolidone (Plasdonet K29-32), talc and magnesium stearate; and a disintegrating layer, comprising levodopa methyl hydrochloride, CD, Eudralack yellow, polyvinylpyrrolidone (Plasdonet K29-32), microcrystalline cellulose (Avicel PH102), croscarmellose sodium (Ac-Di-Sol), talc and magnesium stearate. Bettini et al. (Eur J Pharm Biopharm 2002 March; 53(2):227-32) disclose the three-layered matrix tablet comprising a water swellable layer, a water erodible layer, and a water disintegratable layer for levodopa methyl ester and CD. This tablet contains a release rate-controlling polymer. The three layers are assembled in the monolithic matrix in different relative positions. In one configuration, the tablet can be useful for the reduction of the morning on-off fluctuation, because it provides an early LD plasma peak. In another configuration the tablet may be useful for the afternoon administration, because it avoids end-of-dose deterioration by providing prolonged release of the drugs.

European Patent No. 0253490 discloses a formulation of LD and CD uniformly dispersed in a polymeric matrix consisting of a mixture of two polymers, one of which is water-soluble, such as hydroxypropyl methylcellulose, and the other of which is weakly water soluble polymer, such as polyvinyl acetate/crotonic acid copolymer. This tablet contains a release rate-controlling polymer.

U.S. Pat. No. 4,361,545 discloses a solid, orally administrable pharmaceutical tablet composition for the slow, zero order release of drugs having a water solubility of about 1/5-1/500 (w/w). The formulations are based upon control of active ingredient release from the surface of the tablet via a controlled surface erosion mechanism. This tablet requires a surface controlling compound which has a water solubility of about 1/1-1/40 (w/w); an erosion controlling compound which has a water solubility of about 1/1-1/10 (w/w); a surface activator which is a disintegrating agent for pharmaceutical compositions at which amount the compound is ineffective as a disintegrating agent, a surfactant which is pharmaceutically acceptable in oral compositions, a binder and a die wall lubricant. This tablet contains a disintegrant and a surfactant.

U.S. Pat. No. 5,192,550 discloses an osmotic device for administering a drug, for example LD and/or CD, for treating central nervous system disorders. The device comprises a first composition comprising a pharmaceutical drug carrier and 100 nanograms to 700 milligrams of drug granules.

U.S. Pat. No. 5,266,332 discloses a method for treating Parkinson's disease wherein the method comprises admitting into the patient an osmotic device comprising a drug composition in the compartment comprising 10 ng to 1200 mg of an anti-Parkinson drug and a hydrophilic polymer.

U.S. Pat. No. 5,532,274 and No. 5,624,960 disclose a formulation having controlled liberation of LD and CD during a short release phase comprising a polymer mixture consisting of polyvinyl alcohols.

U.S. Pat. No. 5,840,756 discloses a pharmaceutical composition that comprises levodopa ethyl ester, hydroxypropyl methylcellulose, hydroxypropylcellulose and a carboxyvinyl polymer, and provides an early burst of LD followed by the maintenance of a sustained level of LD. The composition contains a release rate controlling polymer and may optionally further contain CD.

U.S. Pat. No. 6,117,453 discloses a solid pharmaceutical composition comprising an active ingredient, such as LD or CD, which is not in an amorphous form, polyethylene oxide, and the balance consisting of conventional additives, excluding basic components. This composition contains a release rate controlling polymer

U.S. Pat. No. 6,217,905 discloses a dosage form for administering an anti-Parkinson drug to a patient, wherein the dosage form comprises: “(a) a composition comprising 0.10 mg to 750 mg of an anti-Parkinson drug and a pharmaceutically acceptable carrier for the anti-Parkinson drug selected from the group consisting of hydroxypropylcellulose, hydroxypropyl methylcellulose and polyvinylpyrrolidone, which composition in the presence of fluid that contacts the dosage form provides a dispensable anti-Parkinson therapeutic formulation; and wherein the dosage form: (b) provides the anti-Parkinson drug substantially-free of adverse effects for administration in a rate-controlled metered dose per unit time over 24 hours. In one embodiment, the anti-Parkinson drug is a combination of LD and CD. Even though the claims are not limited to an osmotic device, that is the only type of formulation exemplified or described.

U.S. Pat. No. 6,238,699 and its related U.S. Pat. No. 6,756,056 disclose a pharmaceutical tablet comprising a sustained release core layer of CD (25-75 mg), LD (100-400 mg), cellulose ether (80 mg), and microcrystalline cellulose, wherein the sustained release layer is overcoated with an immediate release layer comprising CD (10-25 mg) and LD (50-200 mg), wherein the sustained and immediate release layers are separated by a drug free excipient layer. A bilayer tablet consisting of one layer of sustained release carbidopa-levodopa adjacent to a layer of immediate release carbidopa-levodopa is also disclosed. This tablet contains a release rate-controlling polymer.

U.S. Pat. No. 6,372,254 discloses a press-coated tablet suitable for oral administration, comprising an immediate-release compartment comprising a compressed blend of an active agent, such as LD, and one or more polymers, and an extended-release compartment, formed by press-coating to substantially envelop the immediate-release compartment, and comprising a compressed blend of the active agent, a hydrophilic polymer and hydrophobic material, wherein the tablet exhibits a first order release of the active agent interrupted by a pulsed delivery of the active agent. This tablet contains a release rate-controlling polymer.

U.S. Pat. No. 6,531,153 discloses a pharmaceutical composition comprising a therapeutically effective amount of LD and of CD, dispersed in a hydrophilic matrix, and an organic acid. The hydrophilic matrix generally comprises a gelling substance such as hydroxypropyl methylcellulose. Other gelling components may be used, such as polyvinylpyrrolidone, poly(vinyl alcohol), hydroxypropylcellulose, hydroxymethylcellulose or gelatin, alone or as a mixture. This pharmaceutical composition contains a release rate-controlling polymer.

U.S. Pat. No. 6,607,751 and U.S. Patent Application Publication No. 20040009219 disclose a controlled release pharmaceutical device that comprises pharmaceutically active substances, such as LD or CD, microbial polysaccharide, and uncrosslinked linear polymer, such as cellulose ether. When the delivery device of this invention is administered to the gastrointestinal tract by oral route it comes into contact with an aqueous environment and hydrates forming a gelatinous layer. The device provides sustained or pulsatile delivery of pharmaceutically active substances for a predetermined period of time. The duration, uniformity and continuity of release of the pharmaceutically active agent(s) can be suitably controlled by varying the relative amount of the xanthan gum and HPMC. This formulation contains a release rate-controlling polymer.

PCT International Publication No. WO 98/47491 discloses a matrix tablet for the sustained release of drug, for example LD and CD. This tablet, however, requires two release rate controlling polymers having opposing wettability characteristics.

U.S. Patent Application Publication No. 20030224045 discloses a pharmaceutical dosage form having an immediate release component and a controlled release component comprising: a) an immediate release component comprising a ratio of CD to LD of from about 1:2 to about 1:50 such that the in vitro dissolution rate of the immediate release component according to measurements under the USP paddle method of 50 rpm in 900 ml aqueous buffer at pH 4 at 37 C is from about 10% to about 90% LD released after 30 minutes and from about 50% to about 99% after 1 hour; b) a controlled release component comprising a ratio of CD to LD of from about 1:2 to about 1:50 such that the in vitro dissolution rate of the controlled release component according to measurements under the USP paddle method of 50 rpm in 900 ml aqueous buffer at pH 4 at 37 C is from about 10% to about 40% LD released after 1 hour; from about 25% to about 60% released after 2 hours; from about 40% to about 75% after 4 hours and from about 55% to about 90% after about 6 hours, the in vitro release rate being independent of pH between pH 1.6 and 7.2 and chosen such that the peak plasma level of LD obtained in vivo occurs between 1 and 6 hours after administration of the dosage form. This pharmaceutical dosage form does not comprise a gastro-resistant controlled release component.

U.S. Patent Application Publication No. 20030228360 discloses a pharmaceutical dosage form having an immediate release component and a controlled release component comprising: a) an immediate release component comprising a ratio of CD to LD of from about 1:1 to about 1:50 such that the in vitro dissolution rate of the immediate release component according to measurements under the USP paddle method of 50 rpm in 900 ml aqueous buffer at pH 4 at 37 C is from about 10% to about 99% LD released after 15 minutes and from about 60% to about 99% after 1 hour; b) a controlled release component comprising a ratio of CD to LD of from about 1:1 to about 1:50 such that the in vitro dissolution rate of the controlled release component according to measurements under the USP paddle method of 50 rpm in 900 ml aqueous buffer at pH 4 at 37 C is from about 10% to about 60% LD released after 1 hour; from about 20% to about 80% released after 2 hours; and from about 30% to about 99% after about 6 hours, the in vitro release rate chosen such that the initial peak plasma level of LD obtained in vivo occurs between 0.1 and 6 hours after administration of the dosage form to a patient. This pharmaceutical dosage form does not comprise a controlled release component comprising LD and optionally CD, and an immediate or rapid release component comprising only CD. This pharmaceutical dosage form does not comprise a gastro-resistant controlled release component.

U.S. Patent Application Publication No. 20040013727 discloses a pharmaceutical hydrophilic gel forming matrix formulation comprising one or more active substances, such as active substances for the treatment of Parkinson's disease, and having a prolonged release of said one or more active substances upon exposure to gastrointestinal fluids, characterized in that said release is substantially ion-strength independent. This formulation contains a release rate-controlling polymer.

U.S. Patent Application Publication No. 20040028735 discloses a pharmaceutical formulation for oral administration of a pharmaceutically active compound (such as compounds selected from anti-parkinsonian agents) which includes a tablet core containing an uncoated granulation of a therapeutically effective amount of at least one pharmaceutically active ingredient, an optional surface active agent, an optional pharmaceutically acceptable alkaline agent and a combination of at least one water soluble binder and at least one water insoluble binder, whereby controlled release is achieved by way of the water soluble and water insoluble binders. The binders can be any pharmaceutically acceptable combination of non-toxic water soluble and water insoluble binders such as the following water-soluble polymers, e.g., polyvinyl alcohol, polyvinylpyrrolidone, methylcellulose, hydroxypropyl cellulose, hydroxymethyl cellulose, and the following water-insoluble polymers, e.g., a polymethacrylic acid copolymer such as Eudragit NE30D. This formulation contains release rate-controlling polymeric binders.

U.S. Patent Application Publication No. 20040166159 discloses a pharmaceutical dosage form comprising an immediate release and a controlled release component, wherein said immediate release component and said controlled release component each comprises an AAAD inhibitor, such as CD, and LD in a ratio of from about 1:1 to about 1:50; wherein said immediate release component exhibits an in vitro dissolution profile comprising at least about 10% LD release after 15 minutes and at least about 60% LD release after 1 hour; and wherein said controlled release component exhibits an in vitro dissolution profile comprising from about 10% to about 60% LD release after 1 hour, from about 20% to about 80% LD release after 2 hours, and at least about 30% LD release after 6 hours. The dosage form can optionally contain a cathecol-O-methyltransferase (COMT) inhibitor. This pharmaceutical dosage form does not comprise a controlled release component comprising LD and optionally CD, and an immediate or rapid release component comprising only CD.

Other patents disclosing a controlled release dosage form containing LD and CD include U.S. Patent Application Publication No. 2005/070608 (dry, solid, tablet, or powder formulation of CD and LD which can be mixed with a liquid to form a stable pharmaceutical product); PCT International Publications No. WO 05/041924 (requires a substrate comprising a complex of LD and/or CD and a transport moiety such as sodium lauryl sulfate), No. WO 03/084514 (requires cellulose ether), No. WO 00/15197 (requires immediate release and controlled release components), No. WO 98/18610 (requires an extruded formulation), and No. WO 01/66081 (requires sucrose fatty acid esters as release rate-controlling material); Korean Patent Application No. KR 2003/056474; and European Patent Applications No. EP 324,947 (requires pelletized formulation), and No. EP 253490 (matrix controlled release tablet requiring a two-polymer polymeric matrix).

The prior art controlled release tablets, which include matrix tablets, layered tablets, coated tablets, and osmotic devices, invariably comprise a release rate-controlling polymer or polymeric coat in order to provide a controlled release of CD and LD. In the absence of these materials, however, prior art tablets provide a rapid release of CD and LD.

It would be an improvement in the art to provide a controlled release tablet that provides a controlled release of CD and LD in the absence of a release rate-controlling polymer or coating. Such a tablet would be simpler to manufacture than existing tablets and also would not have the potential for degradation of the CD and LD by the release rate-controlling polymer or coatings or conditions used to include them in the formulation. It would be another improvement in the art to provide oral dosage forms that provide a reduced dosing frequency compared to the oral LD-CD ER dosage forms currently available, and/or a faster relief for patients in the morning off-state.

### SUMMARY OF THE INVENTION

The present invention seeks to overcome the disadvantages of related dosage forms known in the art. In one aspect, the invention provides an extended release solid pharmaceutical composition comprising LD and CD, which are released slowly and substantially continuously over a 1 to 4-hour period when the tablet is placed in an aqueous medium.

Specific embodiments of the invention include those wherein: 1) the extended release composition comprises LD, CD, an organic acid and a carbohydrate or sodium chloride; 2) the extended release composition is included in a tablet; 3) the CD is further included in the tablet in a rapid or immediate release form; 4) the LD is also included in the tablet in a rapid or immediate release form; 5) the LD is included in the tablet in a delayed-extended release form; 6) the tablet further comprises a delayed release coating surrounding the extended release composition or a delayed release material in the controlled release composition such that the extended release of LD and/or CD is delayed by a lag time; 7) the tablet excludes significant amounts of a release rate modifying polymer; 8) the controlled release composition excludes significant amounts of a disintegrant; 9) the controlled release composition excludes significant amounts of a surfactant; and/or 10) the tablet further comprises a finish or polish coat to improve its aesthetic appearance.

Another aspect of the invention provides a LD extended release dosage form comprising: a LD, and optionally CD, extended release composition; and a CD immediate or rapid release composition, such that the LD is released slowly and substantially continuously over a 1 to 4-hour period after exposure of the tablet to an aqueous environment, and the CD is released within about 60 min from the immediate or rapid release composition.

Another aspect of the invention provides a LD delayed and extended dosage form comprising: an extended release composition comprising LD, and optionally CD, surrounded by an enteric coating, and a combination LD-CD immediate or rapid release composition surrounding the enteric coating, such that release of the LD, and optionally the CD, from the extended release composition is delayed and then released slowly and substantially continuously over a 1 to 4-hour period after exposure of the tablet to an aqueous environment, and the combination LD-CD composition is released within about 60 min after initiation of its release.

Yet another aspect of the invention provides a method of treating a disease, disorder or syndrome that is responsive to combination LD and CD therapy, the method comprising the step of orally administering less unit doses as compared to the oral LD-CD ER dosage forms currently available, for example SINEMET CR. This reduction in unit dose requirement, i.e. a reduction in the total number of unit doses per day required to achieve a particular clinical endpoint, is achieved because the formulation of the invention provides an increase in LD bioavailability of about 10% to 90% as compared to that observed for SINEMET CR and/or because the formulation of the invention provides an increase of the mean residence time of LD in the systemic circulation (blood plasma) up to about 30% over that provided by SINEMET CR when administered at the same dosage level and, consequently, provides sustained therapeutic plasma levels above the minimum therapeutic threshold up to about 5 to 12 hours after dosing.

Yet another aspect of the invention provides a method of treating a disease, disorder or syndrome that is responsive to combination LD and CD therapy, the method comprising the step of orally administering to a subject a LD-CD dosage form that releases in the stomach of the subject the first 15 to 40% of the LD dose during the first hour after administration, thereby producing higher plasma levels of LD above the minimum therapeutic threshold as compared to that observed for SINEMET CR when administered in the same dosage amount.

Yet another aspect of the invention provides an oral dosage form that provides a reduction of the required dose of CD, as compared to administration of SINEMET CR in order to achieve about the same therapeutic benefit, by releasing the CD in regions of the gastrointestinal tract having a pH less than or equal to about 5, whereby overall GI absorption of the CD is improved by minimizing its in situ degradation in the GI tract, in other words, by making the CD immediately available for absorption so any exposure to solution (gastric media) above pH 5 is minimized.

Other features, advantages and embodiments of the invention will become apparent to those skilled in the art by the following description, accompanying examples.

### BRIEF DESCRIPTION OF THE FIGURES

The following drawings are part of the present specification and are included to further demonstrate certain aspects of the invention. The invention may be better understood by reference to one or more of these drawings in combination with the detailed description of the specific embodiments presented herein.

FIG. 1 depicts the in vitro release profiles of LD and CD from the exemplary tablets of Examples 1 and 2.

FIG. 2 depicts the in vitro release profiles of LD and CD from the exemplary tablets of Example 8.

FIG. 3 depicts the in vitro release profiles of LD and CD from the exemplary tablets of Example 11.

FIG. 4 depicts the in vitro release profiles of LD and CD from the exemplary tablets of Example 12.

FIG. 5 depicts the LD mean plasma concentration vs. time curves from the exemplary tablets of Example 11 and Sinemet CR administered according to Example 13.

FIG. 6 depicts the CD mean plasma concentration vs. time curves from the exemplary tablets of Example 11 and Sinemet CR administered according to Example 13.

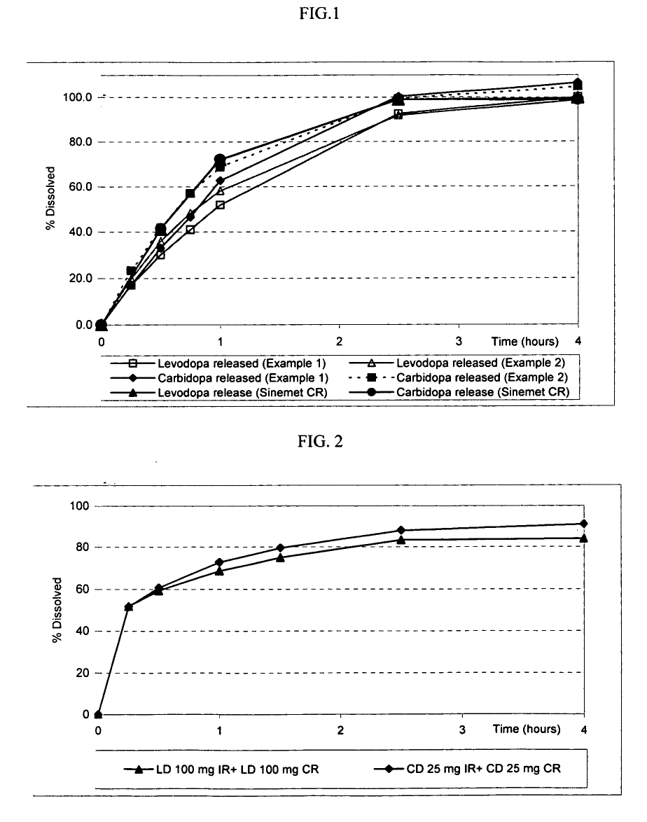

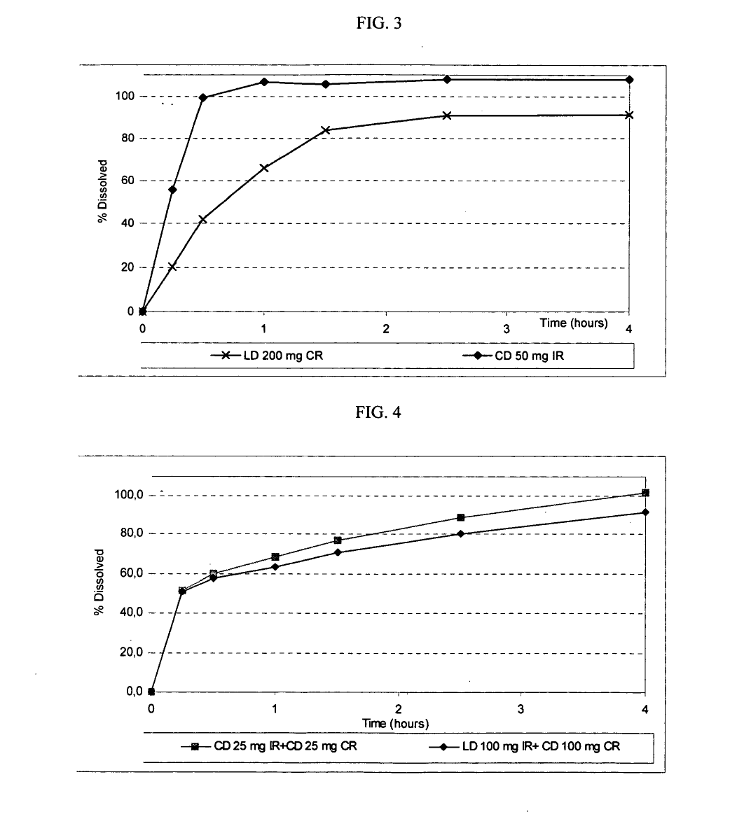

### DETAILED DESCRIPTION OF THE INVENTION

The present invention provides a tablet for the oral administration of LD and CD to a patient suffering from a movement related disease, disorder or syndrome. An orally administrable solid pharmaceutical composition comprising LD in controlled release form and CD in controlled and/or rapid release form is provided. The pharmaceutical composition optionally includes LD in immediate or rapid release form.

The invention may be better understood by reference to the following definitions provided herein.

The term “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A “therapeutically effective amount” is the amount or quantity of drug, which is sufficient to elicit the required or desired therapeutic response, or in other words, the amount that is sufficient to elicit an appreciable biological response when administered to a patient.

By “immediate release” (IR) is meant a release of an active agent to an environment over a period of seconds to no more than about 30 minutes once release has begun and release begins within a second to no more than about 15 minutes after exposure to an aqueous environment. An immediate release composition releases drug in the buccal cavity, esophagus and/or stomach.

By “rapid release” (RR) is meant a release of an active agent to an environment over a period of 159 minutes once release has begun and release can begin within a few minutes after exposure to an aqueous environment or after expiration of a delay period (lag time) after exposure to an aqueous environment. In general, a rapid release composition releases drug in the stomach, jejunum or duodenum after oral administration, provided the composition does not include a delayed release material or delayed release coating. In such a case, the rapid release composition would release drug in the upper, middle and/or lower intestine or colon.

By “extended release” (ER) is meant a controlled release of an active agent from a dosage form to an environment over (throughout or during) an extended period of time, e.g. greater than or equal to one hour. As used herein, the term “extended release” profile assumes the definition as widely recognized in the art of pharmaceutical sciences. An extended release dosage form will release drug at substantially constant rate over an extended period of time or a substantially constant amount of drug will be released incrementally over an extended period of time. The term “extended release”, as regards to drug release, includes the terms “controlled release”, “prolonged release”, “sustained release”, or “slow release”, as these terms are used in the pharmaceutical sciences.

By “controlled release” (CR) is meant a release of an active agent to an environment over a period of about eight hours up to about 12 hours, 16 hours, 18 hours, 20 hours, a day, or more than a day. A controlled release can begin within a few minutes after exposure to an aqueous environment or after expiration of a delay period (lag time) after exposure to an aqueous environment.

By “sustained release” (SR) is meant a controlled release of an active agent to maintain a constant drug level in the blood or target tissue of a subject to which the pharmaceutical composition is administered.

As used herein, a “dosage form” is a solid dosage form containing the pharmaceutical composition of the invention and being suitable for oral administration to a patient (subject).

A “zero-order” release profile characterizes the release profile of a dosage form that releases a constant amount of drug per unit time. A “pseudo-zero order” release profile is one that approximates a zero-order release profile.

A “first order” release profile characterizes the release profile of a dosage form that releases a constant percentage of an initial drug charge per unit time. A “pseudo-first order” release profile is one that approximates a first order release profile.

A delayed but controlled or extended release dosage form is one that provides a delayed release of a drug followed by a controlled or extended release of the drug. By delayed release is meant any formulation technique wherein release of the active substance from the dosage form is modified to occur at a later time than that from a conventional immediate release product. In other words, the beginning of the controlled release of drug is delayed by an initial period of time. The period of delay is generally about 5 minutes to 10 hours, or 30 minutes to 5 hours, or 1 hour to 3 hours.

As used herein, the term “release rate-controlling coating” refers to a coating surrounding a tablet that controls the rate of release of drug from an associated composition such that the drug is released substantially continuously over an extended period of time.

A delayed release coating is not a release-rate controlling coating, since a delayed release coating does not control the rate of drug release. A delayed release coating merely delays the initial release of drug from an associated composition. The present pharmaceutical composition can include a delayed release coating that delays the initial release of CD and/or LD from a controlled release composition or a rapid release composition. The present pharmaceutical composition can also include a delayed release material in a controlled release composition or a rapid release composition such that the delayed release material delays the initial release of CD and/or LD from the controlled release composition or the rapid release composition.

The term “AUC” refers to the area under the plasma concentration-time curve, as calculated by the trapezoidal rule over the complete dosing interval.

The term “Cmax” refers to the highest plasma concentration of the drug attained within the dosing interval.

The term “Tmax” refers to the time period which elapses after administration of the dosage form at which the plasma concentration of the drug attains the highest plasma concentration of drug attained within the dosing interval. By “shorter Tmax” applicants refer to the absorption of active agent(s) at earlier time points than would be found using other dosage forms.

The pharmaceutical composition of the invention can include a wide range of different excipients. Suitable types of excipients include adsorbents, antioxidants, acidifying agent, alkalizing agent, buffering agents, colorants, flavorants, sweetening agents, tablet antiadherents, tablet binders, tablet diluents, tablet direct compression excipients, tablet disintegrants, tablet glidants, tablet lubricants, tablet opaquants and/or tablet polishing agents. Similar excipients used in capsule formulations can also be include in the present pharmaceutical composition.

As used herein, the term “alkalizing agent” is intended to mean a compound used to provide alkaline medium for product stability. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, diethanolamine, organic amine base, alkaline amino acids and trolamine and others known to those of ordinary skill in the art.

As used herein, the term “acidifying agent” is intended to mean a compound used to provide an acidic medium for product stability. Such compounds include, by way of example and without limitation, acetic acid, acidic amino acids, citric acid, fumaric acid and other alpha hydroxy acids, hydrochloric acid, ascorbic acid, phosphoric acid, sulfuric acid, tartaric acid and nitric acid and others known to those of ordinary skill in the art.

As used herein, the term “adsorbent” is intended to mean an agent capable of holding other molecules onto its surface by physical or chemical (chemisorption) means. Such compounds include, by way of example and without limitation, powdered and activated charcoal and other materials known to one of ordinary skill in the art.

As used herein, the term “antioxidant” is intended to mean an agent that inhibits oxidation and thus is used to prevent the deterioration of preparations by the oxidative process. Such compounds include, by way of example and without limitation, ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate and sodium metabisulfite and other materials known to one of ordinary skill in the art.

As used herein, the term “buffering agent” is intended to mean a compound used to resist change in pH upon dilution or addition of acid or alkali. Such compounds include, by way of example and without limitation, potassium metaphosphate, potassium phosphate, monobasic sodium acetate and sodium citrate anhydrous and dihydrate and other materials known to one of ordinary skill in the art.

As used herein, the term “sweetening agent” is intended to mean a compound used to impart sweetness to a preparation. Such compounds include, by way of example and without limitation, aspartame, dextrose, glycerin, mannitol, saccharin sodium, sorbitol and sucrose and other materials known to one of ordinary skill in the art.

As used herein, the term “antiadherent” is intended to mean an agent that prevents the sticking of tablet formulation ingredients to punches and dies in a tableting machine during production. Such. compounds include, by way of example and without limitation, magnesium stearate, talc, calcium stearate, glyceryl behenate, polyethylene glycol (PEG), hydrogenated vegetable oil, mineral oil, stearic acid and other materials known to one of ordinary skill in the art.

As used herein, the term “binder” is intended to mean a substance used to cause adhesion of powder particles in granulations. Such compounds include, by way of example and without limitation, acacia, poly(vinylpyrrolidone), compressible sugar (e.g., NuTab), ethylcellulose, gelatin, liquid glucose, povidone, pregelatinized starch, tragacanth, starch, cellulose materials such as methyl cellulose and sodium carboxy methyl cellulose, alginic acids and salts thereof, polyethylene glycol, guar gum, polysaccharide, bentonites, sugars, invert sugars, poloxamers (PLURONIC™ F68, PLURONIC™ F127), collagen, albumin, cellulosics in nonaqueous solvents, combinations thereof and the like. Other binders include, for example, polypropylene glycol, polyoxyethylene-polypropylene copolymer, polyethylene ester, polyethylene sorbitan ester, polyethylene oxide, combinations thereof and other materials known to one of ordinary skill in the art.

As used herein, the term “diluent” or “filler” is intended to mean inert substances used as fillers to create the desired bulk, flow properties, and compression characteristics in the preparation of tablets and capsules. Such compounds include, by way of example and without limitation, dibasic calcium phosphate, kaolin, lactose, sucrose, mannitol, microcrystalline cellulose, powdered cellulose, precipitated calcium carbonate, sorbitol, and starch and other materials known to one of ordinary skill in the art.

As used herein, the term “direct compression excipient” is intended to mean a compound used in direct compression tablet formulations. Such compounds include, by way of example and without limitation, dibasic calcium phosphate (e.g., Ditab) and other materials known to one of ordinary skill in the art.

As used herein, the term “glidant” is intended to mean an agent used in tablet and capsule formulations to promote flowability of the granulation. Such compounds include, by way of example and without limitation, colloidal silica, cornstarch, talc, calcium silicate, magnesium silicate, colloidal silicon, silicon hydrogel and other materials known to one of ordinary skill in the art.

As used herein, the term “lubricant” is intended to mean a substance used in the instant formulations to reduce friction during compression or other processing. Such compounds include, by way of example and without limitation, calcium stearate, magnesium stearate, mineral oil, stearic acid, and zinc stearate and other materials known to one of ordinary skill in the art.

As used herein, the term “opaquant” is intended to mean a compound used to render a capsule or a tablet coating opaque. May be used alone or in combination with a colorant. Such compounds include, by way of example and without limitation, titanium dioxide, talc and other materials known to one of ordinary skill in the art.

As used herein, the term “polishing agent” is intended to mean a compound used to impart an attractive sheen to coated tablets. Such compounds include, by way of example and without limitation, camauba wax, white wax and other materials known to one of ordinary skill in the art.

As used herein, the term “disintegrant” is intended to mean a compound used in solid dosage forms to promote the disruption of the solid mass into smaller particles that are more readily dispersed or dissolved. Exemplary disintegrants include, by way of example and without limitation, starches such as corn starch, potato starch, pre-gelatinized and modified starches thereof, sweeteners, clays, such as bentonite, microcrystalline cellulose (e.g., Avicel), carboxymethylcellulose calcium, cellulose polyacrilin potassium (e.g., Amberlite), alginates, sodium starch glycolate, gums such as agar, guar, locust bean, karaya, pectin, tragacanth; crospovidone and other materials known to one of ordinary skill in the art.

As used herein, the term “colorant” is intended to mean a compound used to impart color to solid (e.g., tablets) pharmaceutical preparations. Such compounds include, by way of example and without limitation, FD&C Red No. 3, FD&C Red No. 20, FD&C Yellow No. 6, FD&C Blue No. 2, D&C Green No. 5, D&C Orange No. 5, D&C Red No. 8, caramel, and ferric oxide, red, other F.D. & C. dyes and natural coloring agents such as grape skin extract, beet red powder, beta-carotene, annato, carmine, turmeric, paprika, and other materials known to one of ordinary skill in the art. The amount of coloring agent used will vary as desired.

As used herein, the term “flavorant” is intended to mean a compound used to impart a pleasant flavor and often odor to a pharmaceutical preparation. Exemplary flavoring agents or flavorants include synthetic flavor oils and flavoring aromatics and/or natural oils, extracts from plants, leaves, flowers, fruits and so forth and combinations thereof. These may also include cinnamon oil, oil of wintergreen, peppermint oils, clove oil, bay oil, anise oil, eucalyptus, thyme oil, cedar leave oil, oil of nutmeg, oil of sage, oil of bitter almonds and cassia oil. Other useful flavors include vanilla, citrus oil, including lemon, orange, grape, lime and grapefruit, and fruit essences, including apple, pear, peach, strawberry, raspberry, cherry, plum, pineapple, apricot and so forth. Flavors that have been found to be particularly useful include commercially available orange, grape, cherry and bubble gum flavors and mixtures thereof. The amount of flavoring may depend on a number of factors, including the organoleptic effect desired. Flavors will be present in any amount as desired by those of ordinary skill in the art. Particular flavors are the grape and cherry flavors and citrus flavors such as orange.

The present pharmaceutical composition can also employ one or more commonly known surface active agents or cosolvents that improve wetting or disintegration of the tablet core or layers.

Plasticizers can also be included in the pharmaceutical composition to modify the properties and characteristics of the polymers used in the coats or core of the composition. As used herein, the term “plasticizer” includes all compounds capable of plasticizing or softening a polymer or binder used in invention. The plasticizer should be able to lower the melting temperature or glass transition temperature (softening point temperature) of the polymer or binder. Plasticizers, such as low molecular weight PEG, generally broaden the average molecular weight of a polymer in which they are included thereby lowering its glass transition temperature or softening point. Plasticizers also generally reduce the viscosity of a polymer. It is possible the plasticizer will impart some particularly advantageous physical properties to the tablet of the invention.

Plasticizers useful in the invention can include, by way of example and without limitation, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, low molecular weight poly(ethylene glycol), citrate ester-type plasticizers, triacetin, propylene glycol and glycerin. Such plasticizers can also include ethylene glycol, 1,2-butylene glycol, 2,3-butylene glycol, styrene glycol, diethylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutylsebacate, acetyltributylcitrate, triethyl citrate, acetyl triethyl citrate, tributyl citrate and allyl glycolate. All such plasticizers are commercially available from sources such as Aldrich or Sigma Chemical Co. It is also contemplated and within the scope of the invention, that a combination of plasticizers may be used in the present formulation. The PEG based plasticizers are available commercially or can be made by a variety of methods, such as disclosed in Poly(ethylene glycol) Chemistry: Biotechnical and Biomedical Applications (J. M. Harris, Ed.; Plenum Press, NY) the disclosure of which is hereby incorporated by reference.

The compositions of the invention can also include oils, for example, fixed oils, such as peanut oil; sesame oil, cottonseed oil, corn oil and olive oil; fatty acids, such as oleic acid, stearic acid and isotearic acid; and fatty acid esters, such as ethyl oleate, isopropyl myristate, fatty acid glycerides and acetylated fatty acid glycerides. It can also be mixed with alcohols, such as ethanol, isopropanol, hexadecyl alcohol, glycerol and propylene glycol; with glycerol ketals, such as 2,2-dimethyl-1,3-dioxolane-4-methanol; with ethers, such as poly(ethyleneglycol) 450, with petroleum hydrocarbons, such as mineral oil and petrolatum; with water, or with mixtures thereof; with or without the addition of a pharmaceutically suitable surfactant, suspending agent or emulsifying agent.

Soaps and synthetic detergents may be employed as surfactants and as vehicles for detergent compositions. Suitable soaps include fatty acid alkali metal, ammonium, and triethanolamine salts. Suitable detergents include cationic detergents, for example, dimethyl dialkyl ammonium halides, alkyl pyridinium halides, and alkylamine acetates; anionic detergents, for example, alkyl, aryl and olefin sulfonates, alkyl, olefin, ether and monoglyceride sulfates, and sulfosuccinates; nonionic detergents, for example, fatty amine oxides, fatty acid alkanolamides, and poly(oxyethylene)-block-poly(oxypropylene) copolymers; and amphoteric detergents, for example, alkyl β-aminopropionates and 2-alkylimidazoline quaternary ammonium salts; and mixtures thereof.

Various other components, not otherwise listed above, can be added to the present formulation for optimization of a desired active agent release profile including, by way of example and without limitation, glycerylmonostearate, nylon, cellulose acetate butyrate, d,l-poly(lactic acid), 1,6-hexanediamine, diethylenetriamine, starches, derivatized starches, acetylated monoglycerides, gelatin coacervates, poly (styrene-maleic acid) copolymer, glycowax, castor wax, stearyl alcohol, glycerol palmitostearate, poly(ethylene), poly(vinyl acetate), poly(vinyl chloride), 1,3-butylene-glycoldimethacrylate, ethyleneglycol-dimethacrylate and methacrylate hydrogels.

It should be understood, that compounds used in the art of pharmaceutical formulation generally serve a variety of functions or purposes. Thus, if a compound named herein is mentioned only once or is used to define more than one term herein, its purpose or function should not be construed as being limited solely to that named purpose(s) or function(s).

The dosage form of the invention can assume any shape or form known in the art of pharmaceutical sciences. The device of the invention can be a capsule, caplet, pill, sphere, tablet, oblong tablet, bar, plate, paraboloid of revolution, ellipsoid of revolution or the like. The dosage form can also include surface markings, cuttings, grooves, letters and/or numerals for the purposes of decoration, identification and/or other purposes.

The dosage form can include a finish coat as is commonly done in the art to provide the desired shine, color, taste or other aesthetic characteristics. Materials suitable for preparing the finish coat are well known in the art and found in the disclosures of many of the references cited and incorporated by reference herein.

The immediate release or rapid release composition includes a water soluble and/or erodible, inert and non-toxic material that is at least partially, and optionally substantially completely, soluble or erodible in an environment of use. Exemplary materials are disclosed in U.S. Pat. No. 4,576,604 to Guittard et al. and U.S. Pat. No. 4,673,405 to Guittard et al., and U.S. Pat. No. 6,004,582 to Faour et al. and the text Pharmaceutical Dosage Forms: Tablets Volume I, 2^{nd} Edition. (A. Lieberman. ed. 1989, Marcel Dekker, Inc.), the relevant disclosures of which are hereby incorporated by reference.

Materials which are suitable for use in the immediate release or rapid release composition include, by way of example and without limitation, water soluble polysaccharide gums such as carrageenan, fucoidan, gum ghatti, tragacanth, arabinogalactan, pectin, and xanthan; water-soluble salts of polysaccharide gums such as sodium alginate, sodium tragacanthin, and sodium gum ghattate; water-soluble hydroxyalkylcellulose wherein the alkyl member is straight or branched of 1 to 7 carbons such as hydroxymethylcellulose, hydroxyethylcellulose, and hydroxypropylcellulose; synthetic water-soluble and low molecular weight cellulose-based lamina formers such as methyl cellulose and its hydroxyalkyl methylcellulose cellulose derivatives such as a member selected from the group consisting of hydroxyethyl methylcellulose, hydroxypropyl methylcellulose, and hydroxybutyl methylcellulose; croscarmellose sodium; other cellulose polymers such as sodium carboxymethylcellulose; and other materials known to those of ordinary skill in the art. Other materials include poly(vinylpyrrolidone), polyvinylalcohol, polyethylene oxide, a blend of gelatin and polyvinyl-pyrrolidone, gelatin, glucose, saccharides, povidone, copovidone, poly(vinylpyrrolidone)-poly(vinyl acetate) copolymer. The artisan of ordinary skill will recognize that the above-noted materials include film-forming polymers that are not release rate controlling materials even though they may include the same chemical fimctionality thereof. This is because film-forming polymers that do not control release rate generally have lower molecular weight than otherwise similar film-forming polymers having higher molecular weight.

A delayed release material (coating) used in the pharmaceutical composition will possess limited solubility or erodibility or be insoluble or non-erodible in a first external fluid, while being soluble and/or erodible in a second external fluid. For example, the delayed release material may be insoluble in the fluid of a first environment of use, such as gastric juices, acidic fluids, or polar liquids, and soluble or erodible in the fluid of a second environment of use, such as intestinal juices, substantially pH neutral or basic fluids, or apolar liquids. A wide variety of other polymeric materials are known to possess these various solubility properties and can be used. Such other polymeric materials include, by way of example and without limitation, cellulose acetate phthalate (CAP), cellulose acetate trimelletate (CAT), poly(vinyl acetate)phthalate (PVAP), hydroxypropyl methylcellulose phthalate (HP), poly(methacrylate ethylacrylate) (1:1) copolymer (MA-EA), poly(methacrylate methylmethacrylate) (1:1) copolymer (MA-MMA), poly(methacrylate methylmethacrylate) (1:2) copolymer, EUDRAGIT™ L-30-D (MA-EA, 1:1), EUDRAGIT™ L-100-55 (MA-EA, 1:1), hydroxypropyl methylcellulose acetate succinate (HPMCAS), COATERIC™ (PVAP), AQUATERIC™ (CAP), AQOAT™ (HPMCAS) and combinations thereof.

An optional polymeric material for the delayed release material/coating is a poly(vinylpyrrolidone)-vinyl acetate copolymer, such as the material supplied by BASF under its KOLLIDON VA64 trademark. This can be mixed with other excipients such as magnesium stearate, povidone, which is supplied by BASF under its KOLLIDON K 30 trademark, and hydroxypropyl methylcellulose, which is supplied by Dow under its METHOCEL E-15 trademark. The materials can be prepared in solutions having different concentrations of polymer according to the desired solution viscosity. For example, a 10% w/v aqueous solution of KOLLIDON™ K 30 has a viscosity of about 5.5-8.5 cps at 20° C., and a 2% w/v aqueous solution of METHOCEL™ E-15 has a viscosity of about 13-18 cps at 20° C.

The delayed release composition can also comprise other materials suitable which are substantially resistant to gastric juices and which will promote enteric release. These materials do not dissolve, disintegrate, or change their structure in the stomach and during the period of time that the dosage form resides in the stomach. Representative materials that keep their integrity in the stomach can comprise a member selected from the group consisting of (a) keratin, keratin sandarac-tolu, salol (phenyl salicylate), salol beta-naphthylbenzoate and acetotannin, salol with balsam of Peru, salol with tolu, salol with gum mastic, salol and stearic acid, and salol and shellac; (b) a member selected from the group consisting of formalized protein, formalized gelatin, and formalized cross-linked gelatin and exchange resins; (c) a member selected from the group consisting of myristic acid-hydrogenated castor oil-cholesterol, stearic acid-mutton tallow, stearic acid-balsam of tolu, and stearic acid-castor oil; (d) a member selected from the group consisting of shellac, ammoniated shellac, ammoniated shellac-salol, shellac-wool fat, shellac-acetyl alcohol, shellac-stearic acid-balsam of tolu, and shellac n-butyl stearate; (e) a member selected from the group consisting of abietic acid, methyl abictate, benzoin, balsam of tolu, sandarac, mastic with tolu, and mastic with tolu, and mastic with acetyl alcohol; (f) acrylic resins represented by anionic polymers synthesized from methacrylate acid and methacrylic acid methyl ester, copolymeric acrylic resins of methacrylic and methacrylic acid and methacrylic acid alkyl esters, copolymers of alkacrylic acid and alkacrylic acid alkyl esters, acrylic resins such as dimethylaminoethylmethacrylate-butylmethacrylate-methylmethacrylate copolymer of 150,000 molecular weight, methacrylic acid-methylmethacrylate 50:50 copolymer of 135,000 molecular weight, methacrylic acid-methylmethacrylate-30:70-copolymer of 135,000 mol. wt., methacrylic acid-dimethylaminoethyl-methacrylate-ethylacrylate of 750,000 mol. wt., methacrylic acid-methylmethacrylate-ethylacrylate of 1,000,000 mol. wt., and ethylacrylate-methylmethacrylate-ethylacrylate of 550,000 mol. wt; and, (g) an enteric composition comprising a member selected from the group consisting of cellulose acetyl phthalate, cellulose diacetyl phthalate, cellulose triacetyl phthalate, cellulose acetate phthalate, hydroxypropyl methylcellulose phthalate, sodium cellulose acetate phthalate, cellulose ester phthalate, cellulose ether phthalate, methylcellulose phthalate, cellulose ester-ether phthalate, hydroxypropyl cellulose phthalate, alkali salts of cellulose acetate phthalate, alkaline earth salts of cellulose acetate phthalate, calcium salt of cellulose acetate phthalate, ammonium salt of hydroxypropyl methylcellulose phthalate, cellulose acetate hexahydrophthalate, hydroxypropyl methylcellulose hexahydrophthalate, polyvinyl acetate phthalate (such as SURETERIC™ of Colorcon), diethyl phthalate, dibutyl phthalate, dialkyl phthalate wherein the alkyl comprises from 1 to 7 straight and branched alkyl groups, aryl phthalates, and other materials known to one or ordinary skill in the art.

Solutes can be added to the tablet. These solutes can aid in either the suspension or dissolution of drug. Exemplary solutes include organic and inorganic compounds such as salts, acids, bases, chelating agents, sodium chloride, lithium chloride, magnesium chloride, magnesium sulfate, lithium sulfate, potassium chloride, sodium sulfite, calcium bicarbonate, sodium sulfate, calcium sulfate, calcium lactate, d-mannitol, urea, tartaric acid, raffinose, sucrose, alpha-d-lactose monohydrate, glucose, combinations thereof and other similar or equivalent materials which are widely known in the art.

Carbidopa (CDP; L-α-hydrazino-α-methyl-β-(3,4-dihydroxybenzene)propanoic acid monohydrate) is commercially available in pharmaceutical grade bulk quantities from sources such as Teva Pharmaceutical Industries LTD (Netanya, Israel), and Divis Laboratories LTD (Andhra Pradesh, India). As used herein, the term CD is intended to mean the crystalline or amorphous form of the anhydrous or hydrate form of the drug.

Levodopa (LD; L-α-amino-α-methyl-β-(3,4-dihydroxybenzene)propanoic acid) is commercially available in pharmaceutical grade bulk quantities from sources such as Divis Laboratories LTD (Andhra Pradesh, India) and Egis Pharmaceuticals LTD (Budapest, Hungary). As used herein, the term LD is intended to mean the crystalline or amorphous form of the anhydrous or hydrate form of the drug.

FIG. 1 depicts the release profiles for formulations prepared as disclosed in Examples 1 and 2 respectively, as compared to SINEMET CR. The in vitro testing was performed with USP Type II dissolution apparatus (paddles), in 900 ml of HCl 0.1N with a fixed agitation rate of 50 revolutions per minute, maintained at a temperature of 37±0.5° C. The samples were tested by high pressure liquid chromatography. The CD release profile for two tablets of Examples 1 and 2 are described as follows. The time is measured as from the instant that the tablet is initially placed in an aqueous environment,

| | | | | |
|---|---|---|---|---|
| | (%) Release | | (%) Release | |
| Time | Example 1 | Average | Example 2 | Average |

| (hrs) | #1 | #2 | (%) | #1 | #2 | (%) |
|---|---|---|---|---|---|---|
| 0 | 0.0 | 0.0 | 0.0 | 0.0 | 0.0 | 0.0 |
| 0.25 | 15.1 | 19.2 | 17.2 | 24.5 | 22.1 | 23.3 |
| 0.5 | 31.1 | 34.9 | 33.0 | 43.2 | 40.2 | 41.7 |
| 0.75 | 44.3 | 48.4 | 46.4 | 58.3 | 55.7 | 57.0 |
| 1 | 59.8 | 65.5 | 62.7 | 70.9 | 66.6 | 68.7 |
| 2.5 | 96.8 | 103.9 | 100.4 | 99.6 | 99.2 | 99.4 |
| 4 | 106.4 | 106.9 | 106.7 | 106.9 | 102.8 | 104.9 |

The LD release profiles for two tablets of Examples 1 and 2 are described as follows:

| | | | | |
|---|---|---|---|---|
| | (%) Release | | (%) Release | |
| Time | (Example 1) | Average | (Example 2) | Average |

| (hrs) | #1 | #2 | (%) | #1 | #2 | (%) |
|---|---|---|---|---|---|---|
| 0 | 0.0 | 0.0 | 0.0 | 0.0 | 0.0 | 0.0 |
| 0.25 | 16.0 | 18.2 | 17.1 | 20.4 | 19.1 | 19.7 |
| 0.5 | 28.8 | 31.6 | 30.2 | 37.3 | 34.6 | 35.9 |
| 0.75 | 38.8 | 43.1 | 40.9 | 49.2 | 47.3 | 48.3 |
| 1 | 48.6 | 55.1 | 51.9 | 59.1 | 57.2 | 58.2 |
| 2.5 | 90.7 | 94.4 | 92.6 | 92.6 | 91.1 | 91.9 |
| 4 | 99.0 | 101.1 | 100.1 | 99.3 | 98.3 | 98.8 |

The LD and CD release profiles for two tablets of SINEMET CR (50 mg CD-200 mg LD) are described as follows:

| | (%) LD Release | | | (%) CD Release | | |
|---|---|---|---|---|---|---|
| Time | (SINEMET CR) | | Average | (SINEMET CR) | | Average |
| (hrs) | #1 | #2 | (%) | #1 | #2 | (%) |
| 0 | 0.0 | 0.0 | 0.0 | 0.0 | 0.0 | 0.0 |
| 0.5 | 41.6 | 40.8 | 41.2 | 41.8 | 40.7 | 41.3 |
| 1 | 72.3 | 71.7 | 72.0 | 72.8 | 71.7 | 72.2 |
| 2.5 | 100.1 | 97.7 | 98.9 | 100.4 | 97.8 | 99.1 |
| 4 | 100.1 | 99.1 | 99.6 | 99.9 | 97.8 | 98.9 |

In standard dissolution assays, the values can vary depending upon the conditions employed. Moreover, the values may have an absolute standard deviation (STD) of ±10%, ±5% time point.

The dissolution profile for the drugs will vary according to the specific formulations used to create the dosage form. The dosage forms of Ex. 1 and Ex. 2 comprise: a single ER composition comprising a combination of LD and CD, wherein the composition excludes a release rate-controlling polymer, and a disintegrant. These dosage forms do not comprise a release rate-controlling coating, nor an IR or RR coating. The dosage form of Ex. 3 comprises: an ER composition comprising LD, (which excludes a release rate-controlling polymer and a disintegrant), an enteric coating surrounding the ER composition, which coating delays the release of the active agents from the ER composition; and an IR or RR coating comprising a combination of LD and CD. The dosage form of Ex. 4 comprises: an ER composition comprising a combination of LD and CD (which exclude a release rate-controlling polymer and a disintegrant), and an IR or RR coating surrounding the ER composition, the coating comprising CD. The dosage form of Ex. 5 comprises: an ER composition comprising LD, (which composition excludes a release rate-controlling polymer and a disintegrant), and an IR or RR coating comprising CD, wherein the coating surrounds the ER composition. The dosage form of Ex. 6 comprises in stacked arrangement: an ER layer comprising a combination of LD and CD, wherein the ER layer includes a release rate-controlling polymer; and an IR or RR layer comprising CD. The dosage form of Ex. 7 comprises in stacked arrangement: an ER layer comprising a combination of LD and CD, (which ER layer excludes a release rate-controlling polymer and a disintegrant) and an IR or RR layer comprising CD. The dosage form of Ex. 8 comprises: an ER layer comprising LD, (which layer includes a release rate-controlling polymer), and an IR or RR layer comprising a combination of LD and CD. The dosage form of Ex. 9 comprises: an ER core composition comprising LD, (which core includes a release rate-controlling polymer), an enteric coating surrounding the core, (wherein the coating delays release of active agent from the core), and an IR or RR coating comprising a combination of LD and CD, wherein the IR or RR coating surrounds the ER coating. The dosage form of Ex. 10 comprises an ER core composition comprising: a combination of LD and CD, (wherein the core excludes a release rate-controlling polymer and a disintegrant), an enteric coating that surrounds the core and delays the release of active agent from the core; and an IR or RR coating comprising a combination of LD and CD, wherein the IR or RR coating surrounds the enteric coating. The dosage form of Ex. 11 comprises in stacked arrangement, an ER layer comprising LD and a release rate-controlling polymer; and an IR or RR layer comprising CD. The dosage form of Ex. 12 comprises in stacked arrangement: an ER layer comprising a combination of LD and CD, (wherein the ER layer excludes a release rate-controlling polymer and a disintegrant), and an IR or RR layer comprising a combination of LD and CD.

While not wishing to be bound by any particular theory, it is believed that the oral dosage forms of the present invention would provide a reduced dosing frequency as compared to the oral LD-CD ER dosage forms that are currently commercially available, e.g. SINEMET CR, by providing first the release of CD to produce an early AADC inhibition at the gastro-intestinal level prior to the absorption of the LD. Consequently, the LD released later than the CD would reach absorption sites and systemic circulation once the AADC is fully inhibited. This synchronization of the action time of CD would provide an improvement of about 10% to 90% in early absorption LD bioavailability compared to that observed for SINEMET CR. Dosage forms designed to achieve this goal are disclosed in Examples 4, 5, 6, 7, and 11, which comprise an ER formulation of LD and optionally in combination with CD, and an IR or RR formulation comprising only CD.

While not wishing to be bound by any particular theory, it is believed that the oral dosage forms of the present invention would also provide a faster relief in patients of the morning off-state caused by the reduction in plasma LD concentration that occurs while a patient is sleeping. Parkinson's patients usually awaken in the morning in the off state and must wait for a morning dose of LD to take effect before they can function comfortably. However, the synchronized dosage forms disclosed in Examples 4, 5, 6, 7, and 11, help minimize the symptom since they rapidly make available the first 15 to 40% of the LD dose dissolved during the first hour in the stomach for the absorption, producing high plasma levels of LD above the therapeutic threshold, before any oral LD-CD ER dosage forms currently available. Other dosage forms designed to achieve this goal are disclosed in Examples 3, 8, 9, 10, and 12, each dosage form of which comprises an ER core formulation of LD and optionally in combination with CD, an optional enteric coating which delays the release of the active agents from the core, and an IR or RR coating formulation comprising a combination of LD and CD and surrounding the core.

While not wishing to be bound by any particular theory, it is believed that the oral dosage forms of the present invention would also provide a reduced dosing frequency as compared to the oral LD-CD ER dosage forms currently available by incrementing the mean residence time of LD in the systemic circulation up to about 30% and, consequently, providing sustained plasma levels above the therapeutic threshold up to about 5 to 12 hours after dosing. The dosage forms designed to achieve this goal comprise at least about 40% of the total LD and CD doses in a combined delayed and extended release formulation, which would start the release of the active agents at pH 5.0 or higher, and complete it in 1.0 to 2.5 hours within the absorption window, and the rest of the LD and CD doses in an immediate or rapid release formulation. These dosage forms will provide a first plasma concentration peak (pulse) and, before plasma concentrations fall below the therapeutic threshold, a second plasma concentration peak (pulse), which would extend the overall time period during which therapeutic levels of the LD are present in the plasma, especially as compared to the SINEMET CR dosage form. The second plasma concentration peak would result in an increased AADC inhibition due to the previously absorbed CD; therefore, it would provide an increase of at least 20% in bioavailability compared to the available extended release products, especially SINEMET CR. Dosage forms designed to achieve this goal are disclosed in Examples 3, 9, and 10, each dosage form of which comprises an ER core formulation of LD, an enteric coating surrounding the core, which coating delays the release of the active agents from the core, and an IR or RR coating formulation surrounding the enteric coating and comprising a combination of LD and CD. Moreover, when these formulations are administered with food, the food may produce an additional delay of the second peak due to the increase in gastric-emptying time, thereby providing an additional extension of the overall time period that the CD is present in the plasma at a therapeutic level.

Furthermore, the oral dosage forms of the present invention may also provide a reduction of the daily total oral dose of CD required to achieve a particular clinical endpoint, as compared to the administration of SINEMET CR, by releasing the CD in the regions of the gastrointestinal tract having a pH less than or equal to about 5, thereby providing improved absorption of CD by reduction of its degradation in vivo. Dosage forms designed to achieve this goal are disclosed in Examples 3, 5, 8, 9, and 11, each dosage form of which comprises and ER formulation of only LD, optionally a delayed coating, and an IR or RR coating formulation comprising a combination of CD and optionally LD.

The range of weight ratio of CD to LD in a tablet can vary from 1:1 to 1:50. The ratio can be varied depending upon the disorder being treated and the amount of drug per unit dose. In one embodiment, the weight ratio of CD to LD is about 1 to 4.

A pharmacokinetic study carried out as described in Example 13 showed that the dosage form of Example 11 (Test, T) of the present invention compared to SINEMET CR (Reference, R), provides a bioequivalent AUC, a higher Cmax (T:R, p<0.05) and a shorter Tmax (T:R, p<0.05) for LD. The parameter values are disclosed in the following table.

| | Geometric | | | | | |
|---|---|---|---|---|---|---|
| | Mean | | Ratio | CI 90% | | |
| | Refer- | | T/R | limits | | |
| Parameter | ence | Test | (% Ref) | Lower | Upper | Power |
| AUCinf | 5741.93 | 5852.26 | 101.92 | 95.39 | 108.90 | 0.99989 |
| (ng*hr/ml) | | | | | | |
| Cmax (ng/ml) | 1411.29 | 2006.45 | 142.17 | 123.66 | 163.46 | 0.83996 |
| Tmax (hr) | 3.05 | 2.10 | 68.72 | 48.46 | 97.44 | 0.27818 |

The pharmacokinetic study also showed that the dosage form of Example 11 (Test, T) of the present invention compared to SINEMET CR (Reference, R), provides bioequivalent AUC and Cmax, and a shorter Tmax (T:R, p<0.05) for carbidopa. The parameter values are disclosed in the following table.

| | Geometric | | | | | |
|---|---|---|---|---|---|---|
| | Mean | | Ratio | CI 90% | | |
| | Refer- | | T/R | limits | | |
| Parameter | ence | Test | (% Ref) | Lower | Upper | Power |
| AUCinf | 518.26 | 0.06 | 106.30 | 97.51 | 115.89 | 0.99472 |
| (ng*hr/ml) | | | | | | |
| Cmax (ng/ml) | 78.53 | 0.12 | 112.38 | 102.03 | 123.78 | 0.98367 |
| Tmax (hr) | 4.41 | −0.39 | 67.52 | 53.53 | 85.15 | 0.47531 |

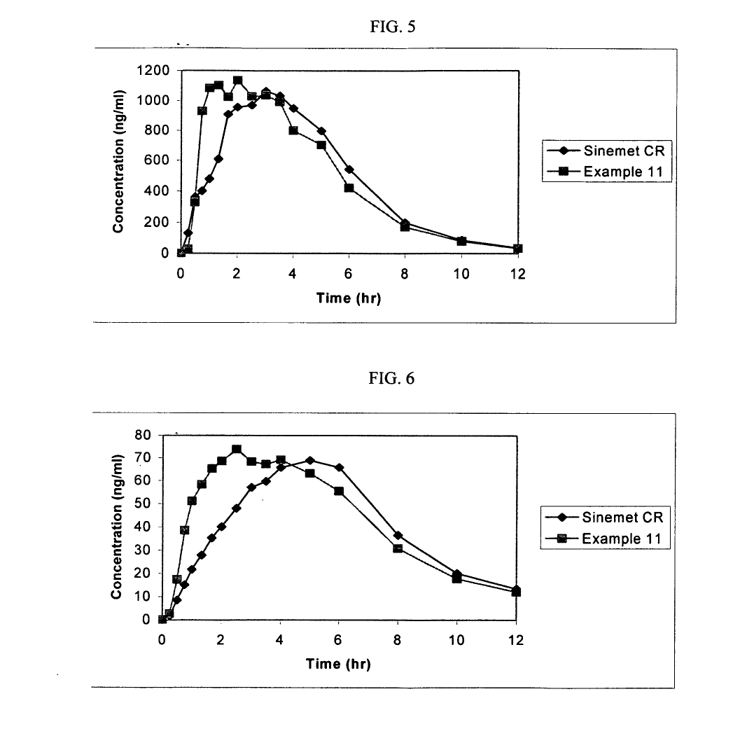

The bilayer tablet of the invention containing an extended release of LD and an immediate or rapid release of CD as compared to the oral LD-CD ER dosage forms that are currently commercially available, e.g. SINEMET CR, provides enhanced absorption of levodopa, shown by an increase on the Cmax between 23.66% and 63.46%, and a faster onset of action, shown by the plasma levels of levodopa and carbidopa provided at earlier time points, in other words, shown by the shorter Tmax. The LD and CD mean plasma concentration vs. time curves from the exemplary tablets of Example 11 and Sinemet CR administered according to Example 13 are disclosed in FIGS. 5 and 6 respectively.

The advantage of the bilayer tablets of the invention containing an extended release of LD and an immediate or rapid release of CD as compared to the oral LD-CD IR dosage forms that are currently commercially available, e.g. SINEMET, is that it provides a reliable and rapid plasma level of LD and a longer duration of action than existing immediate release dosage forms which may offer a rapid plasma level of LD but a short duration of action.

The pharmaceutical composition of the invention is used to treat involuntary movement in any disease, syndrome or other disorder known to be treatable with the combination of CD and LD. Exemplary diseases, syndromes and disorders include Parkinson's disease, Parkinson's disease-like disorders that occur due to injury to or illness in the nervous system, post-encephalitic parkinsonism, symptomatic parkinsonism due to carbon monoxide intoxication and/or manganese intoxication, tremors in complex regional pain syndrome, childhood amblyopia, frontal lobe dysfunction in traumatic brain injury, movement disorder following midbrain haemorrhage, locked-in syndrome, adult age phenylketonuria with extrapyramidal syndrome, progressive supranuclear palsy, restless leg syndrome, dopamine deficiency syndrome, axial muscle rigidity associated with etretinate therapy, tardive dystonia (dyskinesia), L-dopa induced dyskinesia, hereditary extrapyramidal system disease, akinetic-rigidity syndromes, rigid forms of torsion dystonia, Hallevorden-Spatz disease, hyperkinetic syndrome, and other diseases, syndromes or disorders discovered to be treatable by this drug combination.

The invention also discloses dosage forms containing amantadine (AMN), LD and optionally CD. Example 14 discloses an exemplary delayed and extended release tablet that provides a delayed and extended release of LD and CD from an enteric coated extended release core of LD and CD and an immediate release external coating comprising AMN, LD and CD. Example 15 discloses an exemplary bilayered tablet that provides an extended release of LD and CD, and an immediate or rapid release of AMN and CD.

Amantadine can be administered to late-stage Parkinsonian patients as adjunct (add-on or combination) therapy to levodopa for treating dyskinesias. For example, PCT International Publication No. W004/087116 to Vergez et al. discloses a phase II, controlled study in a double-blind setting carried out to evaluate the impact of the combination of amantadine and citalopram in the UPDRS score of patients suffering from motor fluctuations. The study showed clear evidences that amantadine on top of levodopa treatment produced a significant improvement in all of the motor fluctuation-related scores (UPDRS and AIMS) in fluctuating patients. Accordingly, the invention also provides a method of treating Parkinson's disease by orally administering a dosage form comprising AMN, CD and LD such that the combined release profile of amantadine, LD and CD will an improved clinical benefit to a subject to which the dosage form is administered, as compared to administration of a dosage form, such as SINENET CR that excludes AMN, wherein the improved clinical benefit can be improved bioavailability and/or less side effects (nausea, vomiting, and/or appetite loss).

The following examples should not be considered exhaustive, but merely illustrative of only a few of the many embodiments contemplated by the present invention. The methods described herein can be followed to prepare tablets according to the invention.

### EXAMPLE 1

The following procedure was used to prepare an exemplary compressed extended release tablet that provides an extended release of LD and CD, in the absence of a release rate-controlling polymer and a release rate-controlling coating.

| Ingredients (functional category) | Amount (mg) |
|---|---|
| Levodopa | 100.0 |
| Carbidopa | 25.0 |
| Organic acid | 5.0-200.0 |
| Carbohydrate | 5.0-150.0 |
| Antiadherent | 0.0-50.0 |
| Lubricant | 1.0-25.0 |
| Total Weight | 310.0 |

Levodopa, CD and the carbohydrate were first individually screened in a rotary mill with a 991 μm screen, and then mixed with the organic acid previously milled using a hammer mill with a 0020 screen, in a mixer granulator for up to 10 minutes to form a homogenous powder blend. The granulation process was initiated by the gradual addition of a granulating solution containing an antiadherent and purified water to the powder blend, with continuous mixing, to change the consistency of the dry powder ingredients to granules. The wet granulation was dried in a static bed at 50-70° C. or in a fluid bed at 40-60° C. for humidity reduction. Next, the dry granules were milled using a rotary mill with a 1575 μm screen at less than 1200 rpm for size reduction. Then, the lubricant, previously sieved through a 30-mesh screen, was added and mixed for about 5 minutes. This final blend was tabletted to provide the tablets.

### EXAMPLE 2

The following procedure was used to prepare an exemplary compressed extended release tablet that provides an extended release of LD and CD, in the absence of a release rate-controlling polymer and a release rate-controlling coating.

| Ingredients (functional category) | Amount (mg) |
|---|---|
| Levodopa | 100.0 |
| Carbidopa | 25.0 |
| Organic acid | 5.0-200.0 |
| Sodium chloride | 5.0-150.0 |
| Antiadherent | 0.0-50.0 |
| Lubricant | 1.0-10.0 |
| Total Weight | 310.0 |

LD and CD were first individually screened in a rotary mill with a 991 μm screen, and then mixed with the organic acid and the sodium chloride previously milled using a hammer mill with a 0020 screen, in a mixer granulator for up to 10 minutes to form a homogenous powder blend. The granulation process was initiated by the gradual addition of a granulating solution containing an antiadherent and purified water to the powder blend, with continuous mixing, to change the consistency of the dry powder ingredients to granules. The wet granulation was dried in a static bed at 50-70° C. or in a fluid bed at 40-60° C. for humidity reduction. Next, the dry granules were milled using a rotary mill with a 1575 μm screen at less than 1200 rpm for size reduction. Then, the lubricant, previously sieved through a 30-mesh screen, was added and mixed for about 5 minutes. This final blend was tabletted to provide the tablets.

### EXAMPLE 3

The following procedure is used to prepare an exemplary compressed extended release tablet that provides a delayed and controlled release of LD, in the presence of a delayed release coating, and an immediate release of LD and CD in an external coating.

| Ingredients (functional category) | Amount (mg) |
|---|---|
| Core (ER) | |
| Levodopa | 100.0 |
| Organic acid | 5.0-100.0 |
| Carbohydrate or sodium chloride | 5.0-100.0 |
| Antiadherent | 0.0-50.0 |
| Lubricant | 1.0-10.0 |
| Enteric coating (DR) | |
| Hydroxypropyl Methylcellulose Phthalate | 5.0-200.0 |
| Triacetin | 0.1-20.0 |
| Coating (IR/RR) | |
| Levodopa | 100.0 |
| Carbidopa | 50.0 |
| Film forming polymer | 5.0-50.0 |
| Disintegrant | 1.0-10.0 |
| Filler | 0.1-10.0 |
| Plasticizer | 0.1-10.0 |

ER is taken to mean extended release. RR is taken to mean rapid release. IR is taken to mean immediate release. DR is taken to mean delayed release.

The core containing carbohydrate is manufactured as disclosed in Example 1, but in the absence of CD. The core containing sodium chloride is manufactured as disclosed in Example 2, but in the absence of CD. Then, an enteric coating composition is prepared as follows: triacetin is blended in purified water. Hydroxypropyl methylcellulose phthalate is added and mixed thoroughly to form a polymer suspension. This suspension is sprayed onto the cores in a perforated pan coater to obtain coated cores.

An external coating composition is prepared as follows: a film former polymer, a plasticizer, a filler and a disintegrant are blended in purified water. The pH of this blend is adjusted to between 6-8 with sodium phosphate dibasic. Then the LD and the CD are added to the blend, and mixed thoroughly to form a polymer mixture. This mixture is sprayed onto the coated cores in a perforated pan coater to obtain the final tablets.

### EXAMPLE 4

The following procedure is used to prepare an exemplary extended release tablet that provides a controlled release of LD and CD, in the absence of a release rate-controlling polymer and a release rate-controlling coating, and an immediate release of CD.

| Ingredients (functional category) | Amount (mg) |
|---|---|
| Core (ER) | |
| Levodopa | 100.0 |
| Carbidopa | 12.5 |
| Organic acid | 5.0-100.0 |
| Carbohydrate or sodium chloride | 5.0-100.0 |
| Antiadherent | 0.0-50.0 |
| Lubricant | 1.0-10.0 |
| Coating (IR/RR) | |
| Carbidopa | 12.5 |
| Film former polymer | 5.0-50.0 |
| Disintegrant | 1.0-10.0 |
| Filler | 5.0-150.0 |
| Plasticizer | 0.1-10.0 |

The core containing carbohydrate is manufactured as disclosed in Example 1. The core containing sodium chloride is manufactured as disclosed in Example 2. An external coating composition is prepared as follows: a film former polymer, a plasticizer, a filler and a disintegrant are blended in purified water. The pH of this blend is adjusted to between 6-8 with sodium phosphate dibasic. Then the CD is added to the blend, and mixed thoroughly to form a polymer mixture. This mixture is sprayed onto the coated cores in a perforated pan coater to obtain the final tablets.

### EXAMPLE 5

The following procedure is used to prepare an exemplary extended release tablet that provides a controlled release of LD, in the absence of a release rate-controlling polymer and a release rate-controlling coating, and an immediate release of CD.

| Ingredients (functional category) | Amount (mg) |
|---|---|
| Core (ER) | |
| Levodopa | 100.0 |
| Organic acid | 5.0-100.0 |
| Carbohydrate or sodium chloride | 5.0-100.0 |
| Antiadherent | 0.0-50.0 |
| Lubricant | 1.0-10.0 |
| Coating (IR/RR) | |
| Carbidopa | 25.0 |
| Film former polymer | 5.0-50.0 |
| Disintegrant | 1.0-10.0 |
| Filler | 5.0-150.0 |
| Plasticizer | 0.1-10.0 |

The core containing LD and carbohydrate is manufactured as disclosed in Example 1. The core containing LD and sodium chloride is manufacture as disclosed in Example 2. The external coating mixture is manufactured as disclosed in Example 4. This mixture is sprayed onto the coated cores in a perforated pan coater to obtain the final tablets.

### EXAMPLE 6

The following procedure is used to prepare an exemplary bilayer tablet that provides an extended release of LD and CD, and an immediate or rapid release of CD.

| Ingredients | Amount (mg) |
|---|---|
| Layer (ER) | |
| Levodopa | 100.0 |
| Carbidopa | 12.5 |
| Filler | 5.0-100.0 |
| CR polymer | 1.0-20.0 |
| Inorganic colorant 1 | 0.1-2.0 |
| Inorganic colorant 2 | 0.5-5.0 |
| Glidant | 0.2-5.0 |
| Lubricant | 1.0-10.0 |
| Layer (IR/RR) | |
| Carbidopa | 12.5 |
| Filler | 50.0-200.0 |
| Binder | 1.0-20.0 |
| Disintegrant | 1.0-10.0 |
| Glidant | 0.2-5.0 |
| Lubricant | 1.0-10.0 |

The extended release layer composition is prepared as follows: the LD, CD and the CR polymer are first individually screened in a rotary mill with a 991 μm screen, and then mixed with the filler and colorants previously milled using a hammer mill with a 0020 screen, in a mixer granulator for up to 10 minutes to form a homogenous powder blend. Then, the granulation process is initiated by the gradual addition of purified water to the powder blend, with continuous mixing, to change the consistency of the dry powder ingredients to granules. The wet granulation is dried in a static bed at 50-70° C. or in a fluid bed at 40-60° C. for humidity reduction. Next, the dry granules are milled using a rotary mill with a 1575 μm screen at less than 1200 rpm for size reduction. Then, the glidant and the lubricant, previously sieved through a 30-mesh screen, is added and mixed for about 5 minutes, to obtain the granules to manufacture the extended release layer.

The immediate or rapid release composition is prepared as follows: the CD, filler, binder and half of the amount of the disintegrant are first individually screened in a rotary mill with a 991 μm screen, and then mixed in a mixer granulator for up to 10 minutes to form a homogenous powder blend. The granulation process is initiated by the gradual addition of water to the powder blend, with continuous mixing, to change the consistency of the dry powder ingredients to granules. The wet granulation is dried in a static bed at 50-70° C. or in a fluid bed at 40-60° C. for humidity reduction. Next, the dry granules are milled using a rotary mill with a 1575 μm screen at less than 1200 rpm for size reduction. Then, the glidant, lubricant, and the other half of the amount of the disintegrant, previously sieved through a 30-mesh screen, are added and mixed for about 5 minutes.

Next, the extended release layer composition and the immediate or rapid release layer composition are compressed as follows: (120-260) mg of the extended release composition is added to the die and tamped, then it is overlaid with (60-260) mg of the immediate or rapid release composition and the two compositions are pressed to obtain the bilayer tablets.

### EXAMPLE 7

The following procedure is used to prepare an exemplary bilayer tablet that provides an extended release of LD and CD, in the absence of a release rate-controlling polymer, and an immediate or rapid release of CD.

| Ingredients | Amount (mg) |
|---|---|
| Layer (ER) | |
| Levodopa | 100.0 |
| Carbidopa | 12.5 |
| Tartaric Acid | 5.0-100.0 |
| Sodium Chloride | 5.0-100.0 |
| Polyethylene Glycol 4000 | 0.0-50.0 |
| Magnesium Stearate | 1.0-10.0 |
| Layer (IR/RR) | |
| Carbidopa | 12.5 |
| Microcrystalline Cellulose | 50.0-200.0 |
| Povidone | 1.0-20.0 |
| Croscarmellose sodium | 1.0-10.0 |
| Colloidal Silicon Dioxide | 0.2-5.0 |
| Magnesium Stearate | 1.0-10.0 |

The extended release layer composition is prepared as follows. The LD and CD are first individually screened in a rotary mill with a 991 μm screen, and then mixed with the tartaric acid and the sodium chloride previously milled using a hammer mill with a 0020 screen, in a mixer granulator for up to 10 minutes to formn a homogenous powder blend. The granulation process is initiated by the gradual addition of a granulating solution containing polyethylene glycol 4000 and purified water to the powder blend, with continuous mixing, to change the consistency of the dry powder ingredients to granules. The wet granulation is dried in a static bed at 50-70° C. or in a fluid bed at 40-60° C. for humidity reduction. Next, the dry granules are milled using a rotary mill with a 1575 μm screen at less than 1200 rpm for size reduction. Then, the magnesium stearate, previously sieved through a 30-mesh screen, is added and mixed for about 5 minutes.

The immediate or rapid release composition is prepared as follows: the CD, microcrystalline cellulose, povidone and a half croscarmellose sodium are first individually screened in a rotary mill with a 991 μm screen, and then mixed in a mixer granulator for up to 10 minutes to form a homogenous powder blend. The granulation process is initiated by the gradual addition of purified water to the powder blend, with continuous mixing, to change the consistency of the dry powder ingredients to granules. The wet granulation is dried in a static bed at 50-70° C. or in a fluid bed at 40-60° C. for humidity reduction. Next, the dry granules are milled using a rotary mill with a 1575 μm screen at less than 1200 rpm for size reduction. Then, colloidal silicon dioxide, the other half of the croscarmellose sodium and magnesium stearate, previously sieved through a 30-mesh screen, are added and mixed for about 5 minutes.

Next, the extended release layer composition and the immediate or rapid release composition are compressed as follows: (120-380) mg of the extended release composition is added to the die and tamped, then it is overlaid with (60-260) mg of the immediate or rapid release composition and the two compositions are pressed to obtain the bilayer tablets.

### EXAMPLE 8

The following procedure was used to prepare a bilayer tablet that provides an extended release of LD and an immediate or rapid release of LD and CD.

| Ingredients | Amount (mg) |
|---|---|
| Layer (ER) | |
| Levodopa | 100.0 |
| Microcrystalline Cellulose | 10.0-200.0 |
| Hydroxyethylcellulose | 2.0-40.0 |
| Red Ferric Oxide | 0.2-4.0 |
| Yellow Ferric Oxide | 1.0-10.0 |
| Colloidal Silicon Dioxide | 0.4-10.0 |
| Magnesium Stearate | 2.0-20.0 |
| Layer (IR/RR) | |
| Levodopa | 100.0 |
| Carbidopa | 50.0 |
| Microcrystalline Cellulose | 10.0-200.0 |
| Povidone | 2.0-40.0 |
| Croscarmellose sodium | 2.0-20.0 |
| Colloidal Silicon Dioxide | 0.4-10.0 |
| Magnesium Stearate | 2.0-20.0 |

The extended release layer composition was prepared as follows: the LD and hydroxyethylcellulose were first individually screened in a rotary mill with a 991 μm screen, and then mixed with microcrystalline cellulose and colorants previously milled using a hammer mill with a 0020 screen, in a mixer granulator for up to 10 minutes to form a homogenous powder blend. The granulation process was initiated by the gradual addition of purified water to the powder blend, with continuous mixing, to change the consistency of the dry powder ingredients to granules. The wet granulation was dried in a static bed at 50-70° C. or in a fluid bed at 40-60° C. for humidity reduction. Next, the dry granules were milled using a rotary mill with a 1575 μm screen at less than 1200 rpm for size reduction. Then, colloidal silicon dioxide and magnesium stearate, previously sieved through a 30-mesh screen, were added and mixed for about 5 minutes.

The immediate or rapid release composition is prepared as follows: the LD, CD, microcrystalline cellulose, povidone and half of the croscarmellose sodium were first individually screened in a rotary mill with a 991 μm screen, and then mixed in a mixer granulator for up to 10 minutes to form a homogenous powder blend. The granulation process was initiated by the gradual addition of purified water to the powder blend, with continuous mixing, to change the consistency of the dry powder ingredients to granules. The wet granulation was dried in a static bed at 50-70° C. or in a fluid bed at 40-60° C. for humidity reduction. Next, the dry granules were milled using a rotary mill with a 1575 μm screen at less than 1200 rpm for size reduction. Then, colloidal silicon dioxide, magnesium stearate, and half of the croscarmellose sodium, previously sieved through a 30-mesh screen, were added and mixed for about 5 minutes.

Next, the extended release layer composition and the immediate or rapid release composition were compressed as follows: (115-385) mg of the extended release composition was added to the die and tamped, then it was overlaid with (165-245) mg of the immediate or rapid release composition and the two compositions were pressed to obtain the bilayer tablets.

### EXAMPLE 9

The following procedure is used to prepare an exemplary dry coated gastro-resistant tablet that provides a delayed and extended release of levodopa and an immediate or rapid release of levodopa and carbidopa.

A general formulation is disclosed below:

| Ingredients | Amount (mg) |
|---|---|
| Core (ER) | |
| Levodopa | 100.0 |
| Filler | 5.0-100.0 |
| CR polymer | 1.0-20.0 |
| Inorganic colorant 1 | 0.1-2.0 |
| Inorganic colorant 2 | 0.5-5.0 |
| Glidant | 0.2-5.0 |
| Lubricant | 1.0-5.0 |
| Enteric coating (DR) | |
| Enteric film polymer | 5.0-200.0 |
| Plasticizer (optional) | 0.1-20.0 |
| Dry Coating (IR/RR) | |
| Levodopa | 100.0 |
| Carbidopa | 50.0 |
| Filler | 50.0-500.0 |
| Binder | 5.0-50.0 |
| Disintegrant | 5.0-20.0 |
| Glidant | 0.2-5.0 |
| Lubricant | 1.0-5.0 |

A specific formulation is disclosed below:

| Ingredients | Amount (mg) |
|---|---|
| Core (ER) | |
| Levodopa | 100.0 |
| Microcrystalline Cellulose | 5.0-100.0 |
| Hydroxyethylcellulose | 1.0-20.0 |
| Red Ferric Oxide | 0.1-2.0 |
| Yellow Ferric Oxide | 0.5-5.0 |
| Colloidal Silicon Dioxide | 0.2-5.0 |
| Magnesium Stearate | 1.0-5.0 |
| Enteric coating (DR) | |
| Polyvinyl acetate phthalate (Sureteric ™) | 5.0-30.0 |
| Dry Coating (IR/RR) | |
| Levodopa | 100.0 |
| Carbidopa | 50.0 |
| Microcrystalline Cellulose | 50.0-500.0 |
| Povidone | 5.0-50.0 |
| Croscarmellose sodium | 5.0-20.0 |
| Colloidal Silicon Dioxide | 0.2-5.0 |
| Magnesium Stearate | 1.0-5.0 |

The extended release layer composition is prepared as follows: the levodopa and hydroxyethylcellutose are first individually screened in a rotary mill with a 991 μm screen, and then mixed with microcrystalline cellulose and colorants previously milled using a hammer mill with a 0020 screen, in a mixer granulator for up to 10 minutes to form a homogenous powder blend. The granulation process was initiated by the gradual addition of purified water to the powder blend, with continuous mixing, to change the consistency of the dry powder ingredients to granules. The wet granulation was dried in a static bed at 50-70° C. or in a fluid bed at 40-60° C. for humidity reduction. Next, the dry granules were milled using a rotary mill with a 1575 μm screen at less than 1200 rpm for size reduction. Then, colloidal silicon dioxide and magnesium stearate, previously sieved through a 30-mesh screen, were added and mixed for about 5 minutes. This final blend was tabletted to provide the tablet cores.

The enteric coating composition is prepared as follows: Polyvinyl acetate phthalate (Sureteric™ of Colorcon) is blended in purified water to form a polymer suspension. This suspension is sprayed onto the cores in a perforated pan coater to obtain gastro-resistant coated cores.

The immediate or rapid release composition is prepared as follows: the levodopa, carbidopa, microcrystalline cellulose, povidone and half of the croscarmellose sodium are first individually screened in a rotary mill with a 991 μm screen, and then mixed in a mixer granulator for up to 10 minutes to form a homogenous powder blend. The granulation process is initiated by the gradual addition of purified water to the powder blend, with continuous mixing, to change the consistency of the dry powder ingredients to granules. The wet granulation is dried in a static bed at 50-70° C. or in a fluid bed at 40-60° C. for humidity reduction. Next, the dry granules are milled using a rotary mill with a 1575 tm screen at less than 1200 rpm for size reduction. Then, colloidal silicon dioxide, magnesium stearate, and the other half of the croscarmellose sodium, previously sieved through a 30-mesh screen, are added and mixed for about 5 minutes and then press coated onto the gastro-resistant coated cores to obtain dry coated gastro-resistant tablets.

### EXAMPLE 10

The following procedure is used to prepare an exemplary dry coated gastro-resistant tablet that provides a delayed and extended release of levodopa and an immediate or rapid release of levodopa and carbidopa.

| Ingredients | Amount (mg) |
|---|---|
| Core (ER) | |
| Levodopa | 100.0 |
| Tartaric Acid | 5.0-100.0 |
| Sodium Chloride | 5.0-100.0 |
| Polyethylene Glycol 4000 | 0.0-50.0 |
| Magnesium Stearate | 1.0-10.0 |
| Enteric coating (DR) | |
| Hydroxypropyl Methylcellulose Phthalate | 5.0-200.0 |
| Triacetin | 0.1-20.0 |
| Dry Coating (IR/RR) | |
| Levodopa | 100.0 |
| Carbidopa | 50.0 |
| Microcrystalline Cellulose | 50.0-500.0 |
| Povidone | 5.0-50.0 |
| Croscarmellose sodium | 5.0-20.0 |
| Colloidal Silicon Dioxide | 0.2-5.0 |
| Magnesium Stearate | 1.0-10.0 |

The levodopa is first individually screened in a rotary mill with a 991 μm screen, and then mixed with the tartaric acid and the sodium chloride previously milled using a hammer mill with a 0020 screen, in a mixer granulator for up to 10 minutes to form a homogenous powder blend. The granulation process is initiated by the gradual addition of a granulating solution containing polyethylene glycol 4000 and purified water to the powder blend, with continuous mixing, to change the consistency of the dry powder ingredients to granules. The wet granulation is dried in a static bed at 50-70° C. or in a fluid bed at 40-60° C. for humidity reduction. Next, the dry granules are milled using a rotary mill with a 1575 μm screen at less than 1200 rpm for size reduction. Then, the magnesium stearate, previously sieved through a 30-mesh screen, is added and mixed for about 5 minutes. This final blend is tabletted to provide the extended release cores.

Then, an enteric coating composition is prepared as follows: triacetin is blended in purified water and the hydroxypropyl methylcellulose phthalate is added and mixed thoroughly to form a polymer suspension. This suspension is sprayed onto the cores in a perforated pan coater to obtain gastro-resistant coated cores.

The immediate or rapid release composition is prepared as disclosed in Example 9, and then press coated onto the gastro resistance coated cores to obtain dry coated gastro-resistant tablets.

### EXAMPLE 11

The following procedure was used to prepare a bilayer tablet that provides an extended release of LD and an immediate or rapid release of CD.

| Ingredients | Amount (mg) |
|---|---|
| Layer (ER) | |
| Levodopa | 200.0 |
| Microcrystalline Cellulose | 10.0-200.0 |
| Hydroxyethylcellulose | 2.0-40.0 |
| Red Ferric Oxide | 0.2-4.0 |
| Yellow Ferric Oxide | 1.0-10.0 |
| Colloidal Silicon Dioxide | 0.4-10.0 |
| Magnesium Stearate | 2.0-20.0 |
| Layer (IR/RR) | |
| Carbidopa | 50.0 |
| Microcrystalline Cellulose | 100.0-400.0 |
| Povidone | 2.0-40.0 |
| Croscarmellose sodium | 2.0-20.0 |
| Colloidal Silicon Dioxide | 0.4-10.0 |
| Magnesium Stearate | 2.0-20.0 |

The extended release layer composition was prepared as follows: the LD and hydroxyethylcellulose were first individually screened in a rotary mill with a 991 μm screen, and then mixed with microcrystalline cellulose and colorants previously milled using a hammer mill with a 0020 screen, in a mixer granulator for up to 10 minutes to form a homogenous powder blend. The granulation process was initiated by the gradual addition of purified water to the powder blend, with continuous mixing, to change the consistency of the dry powder ingredients to granules. The wet granulation was dried in a static bed at 50-70° C. or in a fluid bed at 40-60° C. for humidity reduction. Next, the dry granules were milled using a rotary mill with a 1575 μm screen at less than 1200 rpm for size reduction. Then, colloidal silicon dioxide and magnesium stearate, previously sieved through a 30-mesh screen, was added and mixed for about 5 minutes.

The immediate or rapid release composition was prepared as follows: the CD, microcrystalline cellulose, povidone and half of the croscarmellose sodium were first individually screened in a rotary mill with a 991 μm screen, and then mixed in a mixer granulator for up to 10 minutes to form a homogenous powder blend. The granulation process was initiated by the gradual addition of purified water to the powder blend, with continuous mixing, to change the consistency of the dry powder ingredients to granules. The wet granulation was dried in a static bed at 50-70° C. or in a fluid bed at 40-60° C. for humidity reduction. Next, the dry granules were milled using a rotary mill with a 1575 μm screen at less than 1200 rpm for size reduction. Then, colloidal silicon dioxide, magnesium stearate, and the other half of the croscarmellose sodium, previously sieved through a 30-mesh screen, were added and mixed for about 5 minutes.

Next, the extended release layer composition and the immediate or rapid release composition were compressed as follows: (216-490) mg of the extended release composition was added to the die and tamped, then it was overlaid with (157-540) mg of the immediate or rapid release composition and the two compositions were pressed to obtain the bilayer tablets.

### EXAMPLE 12

The following procedure was used to prepare a bilayer tablet that provides an extended release of LD and CD in the absence of a release rate-controlling polymer and an immediate or rapid release of LD and CD.

| Ingredients | Amount (mg) |
|---|---|
| Layer (ER) | |
| Levodopa | 50.0 |
| Carbidopa | 12.5 |
| Tartaric Acid | 5.0-100.0 |
| Sodium Chloride | 5.0-100.0 |
| Polyethylene Glycol 4000 | 0.00-50.00 |
| Magnesium Stearate | 1.0-10.0 |
| Layer (IR) | |
| Levodopa | 50.0 |
| Carbidopa | 12.5 |
| Microcrystalline Cellulose | 5.0-100.0 |
| Povidone | 1.0-20.0 |
| Croscarmellose sodium | 1.0-10.0 |
| Colloidal Silicon Dioxide | 0.2-5.0 |
| Magnesium Stearate | 1.0-10.0 |

The extended release layer composition was prepared as follows: the LD and CD were first individually screened in a rotary mill with a 991 μm screen, and then mixed with the organic acid and the sodium chloride previously milled using a harmer mill with a 0020 screen, in a mixer granulator for up to 10 minutes to form a homogenous powder blend. The granulation process was initiated by the gradual addition of a granulating solution containing polyethylene glycol 4000 and purified water to the powder blend, with continuous mixing, to change the consistency of the dry powder ingredients to granules. The wet granulation was dried in a static bed at 50-70° C. or in a fluid bed at 40-60° C. for humidity reduction. Next, the dry granules were milled using a rotary mill with a 1575 μm screen at less than 1200 rpm for size reduction. Then, the magnesium stearate, previously sieved through a 30-mesh screen, was added and mixed for about 5 minutes.

The immediate or rapid release composition is prepare as follows: the LD and CD, microcrystalline cellulose, povidone and half of the croscarmellose sodium were first individually screened in a rotary mill with a 991 μm screen, and then mixed in a mixer granulator for up to 10 minutes to form a homogenous powder blend. The granulation process was initiated by the gradual addition of purified water to the powder blend, with continuous mixing, to change the consistency of the dry powder ingredients to granules. The wet granulation was dried in a static bed at 50-70° C. or in a fluid bed at 40-60° C. for humidity reduction. Next, the dry granules were milled using a rotary mill with a 1575 μm screen at less than 1200 rpm for size reduction. Then, colloidal silicon dioxide, magnesium stearate, and the other half of the croscarmellose sodium, previously sieved through a 30-mesh screen, were added and mixed for about 5 minutes.

Next, the extended release layer composition and the immediate or rapid release composition were compressed as follows: (70-410) mg of the extended release composition was added to the die and tamped, then it was overlaid with (70-210) mg of the immediate or rapid release composition and the two compositions were pressed to obtain the bilayer tablets.

### EXAMPLE 13

A pharmacokinetic study (four ways crossover Williams design) will be carried out to test four treatments, A, B, C, and D. Treatment A is the dosage form of Example 9 of the present invention. Treatment B is the dosage form of Example 11 of the present invention. Treatments C and D contained reference treatments. Treatment C is a single extended release tablet of SFNEMET CR containing 50 mg of CD and 200 mg of LD (R1). And, treatment D is a combination of one SINEMET (IR) tablet of 25 mg of CD and 100 mg of LD plus one SINEMET CR tablet of 25 mg of CD and 100 mg of LD (R2).

Twenty healthy hospitalized subjects (non-smokers, male and females between ages of 21-50) are randomly separated into four equally sized groups, each of them receiving the four formulations in four different sequences according to the Williams design with a washout period of one week before changing the treatment. Blood samples are taken periodically from 0 to 16 hrs after administration and plasma aliquots are obtained immediately and stored ta −20° C. for later analysis by HPLC with electrochemical detection to determine LD and CD content. The following pharmacokinetic parameters are calculated from the plasma concentration curve for each formulation and each subject: area under the curve from 0-48 hrs (AUC₀₋ₜ) and extrapolated to infinity (AUC_{0-inf}); maximum concentration of drug in plasma (Cₘₐₓ); and time to reach Cₘₐₓ (Tₘₐₓ). Safety analysis is performed by evaluating physical examination, vital signs and adverse event records. Statistical comparisons are made using Analysis of Variance (ANOVA) after logarithmic transformation for the crossover design. Geometric least square means and classical 90% confidence intervals for the ratio (test/control) of AUC₀₋ₜ and AUC_{0-inf} are calculated in order to evaluate relative bioavailability.

### EXAMPLE 14

The following procedure is used to prepare an exemplary delayed and extended release tablet that provides a delayed and extended release of LD from an enteric coated extended release core of LD and an immediate release external coating comprising AMN, LD and CD.

| Ingredients (functional category) | Amount (mg) |
|---|---|
| Core (ER) | |
| Levodopa | 100.0 |
| Filler | 5.0-100.0 |
| CR polymer | 1.0-20.0 |
| Inorganic colorant 1 | 0.1-2.0 |
| Inorganic colorant 2 | 0.5-5.0 |
| Glidant | 0.2-5.0 |
| Lubricant | 1.0-5.0 |
| Enteric coating (DR) | |
| Enteric film polymer | 5.0-200.0 |
| Plasticizer | 0.1-20.0 |
| Coating (IR/RR) | |
| Amantadine | 100.0 |
| Levodopa | 100.0 |
| Carbidopa | 50.0 |
| Filler | 25.0-150.0 |
| Binder | 1.5-15.0 |
| Disintegrant | 1.0-30.0 |
| Glidant | 0.5-6.0 |
| Lubricant | 1.0-5.0 |
| Coating (finish coat) | |
| Opadry Y30 18084-A | 5.0-25.0 |
| Colorant | 1.0-1.5 |
| Purified Water | 30.0-290.0 |

The extended release layer composition is prepared as follows: the levodopa and a CR polymer are first individually screened in a rotary mill with a 991 μm screen, and then mixed with the filler and colorants previously milled using a hammer mill with a 0020 screen, in a mixer granulator for up to 10 minutes to form a homogenous powder blend. The granulation process was initiated by the gradual addition of purified water to the powder blend, with continuous mixing, to change the consistency of the dry powder ingredients to granules. The wet granulation was dried in a static bed at 50-70° C. or in a fluid bed at 40-60° C. for humidity reduction. Next, the dry granules were milled using a rotary mill with a 1575 μm screen at less than 1200 rpm for size reduction. Then, the glidant and the lubricant, previously sieved through a 30-mesh screen, were added and mixed for about 5 minutes. This final blend was tabletted to provide the tablet cores.

The enteric coating composition is prepared as follows: a plastizicer is blended in purified water. The enteric film polymer is added and mixed thoroughly to form a polymer suspension. This suspension is sprayed onto the cores in a perforated pan coater to obtain gastro-resistant coated cores.

The immediate or rapid release composition is prepared as follows: arnantadine, LD, CD, a filler, a binder and a disintegrant are placed in a high shear mixer and mix for 5 minutes. The granulation process is initiated by the gradual addition of purified water to the high shear mixer with continuous blending to produce a wet blend. Next, the wet blend is granulated and dried at 40-50° C. for 20 minutes in a fluid bed to remove the water. Then, the dry granules are screened through a 20 USP mesh screen for size reduction. Next, the screened granules are mixed with a glidant and a lubricant, which have been previously passed through a 60 mesh screen, in a V-Blender during 5 minutes. The granulate is applied over the cores through compression to obtain AMN-LD-CD coated gastro resistant cores. The tablets have an outer diameter of about 12 mm.

A finish coat comprising Opadry and a colorant in purified water is applied onto the AMN-LD-CD coated gastro resistant cores to obtain the fmal tablets.

### EXAMPLE 15

The following procedure is used to prepare an exemplary bilayer tablet that provides an extended release of LD and CD, and an immediate or rapid release of AMN and CD.

| Ingredients | Amount (mg) |
|---|---|
| Layer (ER) | |
| Levodopa | 100.0-200.0 |
| Carbidopa | 25.0-50.0 |
| Filler | 5.0-180.0 |
| CR polymer | 1.0-36.0 |
| Inorganic colorant 1 | 0.1-3.6 |
| Inorganic colorant 2 | 0.5-9.0 |
| Glidant | 0.2-9.0 |
| Lubricant | 1.0-5.0 |
| Layer (IR/RR) | |
| Amantadine | 50.0-200.0 |
| Carbidopa | 25.0-50.0 |
| Filler | 50.0-360.0 |
| Binder | 1.0-36.0 |
| Disintegrant | 1.0-18.0 |
| Glidant | 0.2-9.0 |
| Lubricant | 1.0-5.0 |

The extended release layer composition is prepared as follows: the LD, CD and the CR polymer are first individually screened in a rotary mill with a 991 μm screen, and then mixed with the filler and colorants previously milled using a hammer mill with a 0020 screen, in a mixer granulator for up to 10 minutes to form a homogenous powder blend. Then, the granulation process is initiated by the gradual addition of purified water to the powder blend, with continuous mixing, to change the consistency of the dry powder ingredients to granules. The wet granulation is dried in a static bed at 50-70° C. or in a fluid bed at 40-60° C. for humidity reduction. Next, the dry granules are milled using a rotary mill with a 1575 μm screen at less than 1200 rpm for size reduction. Then, the glidant and the lubricant, previously sieved through a 30-mesh screen, is added and mixed for about 5 minutes, to obtain the granules to manufacture the extended release layer.

The immediate or rapid release composition is prepared as follows: the AMN, CD, filler, binder and half of the amount of the disintegrant are first individually screened in a rotary mill with a 991 μm screen, and then mixed in a mixer granulator for up to 10 minutes to form a homogenous powder blend. The granulation process is initiated by the gradual addition of water to the powder blend, with continuous mixing, to change the consistency of the dry powder ingredients to granules. The wet granulation is dried in a static bed at 50-70° C. or in a fluid bed at 40-60° C. for humidity reduction. Next, the dry granules are milled using a rotary mill with a 1575 μm screen at less than 1200 rpm for size reduction. Then, the glidant, lubricant, and the other half of the amount of the disintegrant, previously sieved through a 30-mesh screen, are added and mixed for about 5 minutes.

Next, the extended release layer composition and the immediate or rapid release layer composition are compressed as follows: (132-493) mg of the extended release composition is added to the die and tamped, then it is overlaid with (128-678) mg of the immediate or rapid release composition and the two compositions are pressed to obtain the bilayer tablets.

The above is a detailed description of particular embodiments of the invention. Those of skill in the art should, in light of the present disclosure, appreciate that many changes can be made in the specific embodiments which are disclosed herein and still obtain a like or similar result without departing from the spirit and scope of the invention. All of the embodiments disclosed and claimed herein can be made and executed without undue experimentation in light of the present disclosure.

1. (canceled)

2. (canceled)

3. (canceled)

4. (canceled)

5. (canceled)

6. (canceled)

7. (canceled)

8. (canceled)

9. (canceled)

10. (canceled)

11. A compressed extended release tablet comprising: an extended release composition comprising levodopa; and an immediate or rapid release composition comprising carbidopa, such that the levodopa is released slowly and substantially continuously over a 2 to 4-hour period after exposure of the tablet to an aqueous environment, and the carbidopa is released within about 60 min after exposure of the tablet to an aqueous environment.

12. The tablet of claim 11, wherein the rapid or immediate release composition further comprises levodopa.

13. The tablet of any one of claims 11 or 12 further comprising a delayed release coating surrounding the extended release composition.

14. The tablet of claim 13, wherein the extended release composition further comprises a delayed release material.

15. The tablet of claim 11 or 12, wherein the extended release composition further comprises a delayed release material.

16. The tablet of any claim 11 or 12, wherein all of the levodopa is released within 4 hours after exposure to an aqueous environment.

17. The tablet of claim 16, wherein the levodopa is released at a zero order or pseudo-zero order rate for a period of about 2 to 4 hours after exposure to an aqueous environment.

18. The tablet of claim 16, wherein the levodopa is released at a first order rate for a period of about 2 to 4 hours after exposure to an aqueous environment.

19. (canceled)

20. (canceled)

21. (canceled)

22. (canceled)

23. (canceled)

24. A tablet according to any one of the above claims 11 or 12 wherein the tablet comprises 25-50 mg of CD and 100-200 mg of LD; and the weight ratio of CD to LD is about 1:4.

25. An extended release tablet comprising:
an extended release core comprising the following ingredients in the approximate amounts indicated:

| Ingredients | |
|---|---|
| Core (ER) | Amount (mg) |
| Levodopa | 100.0 |
| Filler | 5.0-100.0 |
| CR polymer | 1.0-20.0 |
| Inorganic colorant 1 | 0.1-2.0 |
| Inorganic colorant 2 | 0.5-5.0 |
| Glidant | 0.2-5.0 |
| Lubricant | 1.0-5.0 |

a delayed release coating surrounding the core; and
a rapid release or immediate release layer surrounding the delayed release coating and comprising the following ingredients in the approximate amounts indicated:

| Ingredients | |
|---|---|
| Dry Coating (IR/RR) | Amount (mg) |
| Levodopa | 100.0 |
| Carbidopa | 50.0 |
| Filler | 50.0-500.0 |
| Binder | 5.0-50.0 |
| Disintegrant | 5.0-20.0 |
| Glidant | 0.2-5.0 |
| Lubricant | 1.0-5.0 |

26. An extended release layered tablet comprising:
an extended release layer comprising the following ingredients in the approximate amounts indicated:

| Ingredients | |
|---|---|
| Layer (ER) | Amount (mg) |
| Levodopa | 100.0 |
| Microcrystalline Cellulose | 10.0-200.0 |
| Hydroxyethylcellulose | 2.0-40.0 |
| Red Ferric Oxide | 0.2-4.0 |
| Yellow Ferric Oxide | 1.0-10.0 |
| Colloidal Silicon Dioxide | 0.4-10.0 |
| Magnesium Stearate | 2.0-20.0 |

and a rapid release or immediate release layer comprising the following ingredients in the approximate amounts indicated:

| Ingredients | |
|---|---|
| Layer (IR/RR) | Amount (mg) |
| Levodopa | 100.0 |
| Carbidopa | 50.0 |
| Microcrystalline Cellulose | 10.0-200.0 |
| Povidone | 2.0-40.0 |
| Croscarmellose sodium | 2.0-20.0 |
| Colloidal Silicon Dioxide | 0.4-10.0 |
| Magnesium Stearate | 2.0-20.0 |

27. An extended release layered tablet comprising:
an extended release layer comprising the following ingredients in the approximate amounts indicated:

| Ingredients | |
|---|---|
| Layer (ER) | Amount (mg) |
| Levodopa | 200.0 |
| Microcrystalline Cellulose | 10.0-200.0 |
| Hydroxyethylcellulose | 2.0-40.0 |
| Red Ferric Oxide | 0.2-4.0 |
| Yellow Ferric Oxide | 1.0-10.0 |
| Colloidal Silicon Dioxide | 0.4-10.0 |
| Magnesium Stearate | 2.0-20.0 |

and a rapid release or immediate release layer comprising the following ingredients in the approximate amounts indicated:

| Ingredients | |
|---|---|
| Layer (IR/RR) | Amount (mg) |
| Carbidopa | 50.0 |
| Microcrystalline Cellulose | 100.0-400.0 |
| Povidone | 2.0-40.0 |
| Croscarmellose sodium | 2.0-20.0 |
| Colloidal Silicon Dioxide | 0.4-10.0 |
| Magnesium Stearate | 2.0-20.0 |

28. (canceled)

29. (canceled)

30. A tablet according to any one of the claims 11, 12, 25-26 or 27, wherein the tablet provides an increase in the mean residence time of LD in the systemic circulation (blood plasma) up to about 30% over that observed for SFNEMET CR.

31. A tablet according to any one of the claims 11, 12, 25-26 or 27, wherein the tablet provides more than a 20% increase on the Cmax as compared to that observed for SINEMET CR.

32. A tablet according to claim 31, wherein the tablet provides more than 30% increase on the Cmax as compared to that observed for SINEMET CR.

33. A tablet according to any one of the claims 11, 12, 25-26 or 27, wherein the tablet provides a shorter Tmax for levodopa and carbidopa as compared to those observed for SINEMET CR.

34. A tablet according to any one of the claims 11, 12 25-26 or 27, wherein the tablet provides a therapeutic plasma level of levodopa for a period of no less than about 5 hours after dosing, wherein the tablet comprises about 50 mg of CD and about 200 mg of LD.

35. (canceled)

36. (canceled)

37. (canceled)

38. (canceled)

39. (canceled)

40. A compressed extended release multi-layered tablet comprising:
(1) an immediate release composition comprising amantadine, levodopa and carbidopa;
(2) an extended release composition comprising levodopa and optionally carbidopa; and
(3) a delayed release composition, wherein the delayed release composition is a delayed release coating surrounding the extended release composition or the delayed release composition is a delayed release material included within the extended release composition, and the levodopa and carbidopa is released substantially continuously over a 1 to 4-hour period after exposure of the tablet to an aqueous environment.

41. A compressed extended release multi-layered tablet comprising:
(1) an immediate release composition comprising amantadine, carbidopa and optionally levodopa; and
(2) an extended release composition comprising levodopa and optionally carbidopa.

42. canceled

43. A tablet according to any one of the claims 11, 12, 25-26 or 27, wherein the tablet provides a therapeutic plasma level of levodopa for a period of no less than about 4 hours after administration, wherein the tablet comprises about 25 mg of CD and about 100 mg of LD.
